(19) United States
(12) Patent Application Publication
(10) Pub. No.: US 2026/0321292 A1
(43) Pub. Date: Oct. 15, 2026

(54) CONTEXT-AWARE TECHNIQUES FOR VIDEO HIGHLIGHT EXTRACTION INTEGRATING AUDIO ANALYSIS, AND SYSTEMS OF USE THEREOF

(71) Applicant: Meta Platforms Technologies, LLC, Menlo Park, CA (US)

(72) Inventors: Akhilesh Kumar, Seattle, WA (US); Shaun Mccarthy, Sammamish, WA (US); Haoyu Ren, San Diego, CA (US); Qinwu Xu, Fremont, CA (US); Andrew Garrod Bosworth, San Mateo, CA (US)

(21) Appl. No.: 19/637,286

(22) Filed: Apr. 2, 2026

Related U.S. Application Data
(60) Provisional application No. 63/795,115, filed on Apr. 25, 2025.
(60) Provisional application No. 63/786,207, filed on Apr. 9, 2025.

(51) Int. Cl. G11B 27/036; A61B 5/00; G06F 3/01; G06V 10/26; G06V 20/40; G10L 17/02; G10L 25/57

(52) U.S. Cl. CPC G11B 27/036; A61B 5/6803; G06F 3/013; G06V 10/26; G06V 20/41; G06V 20/49; G10L 17/02; G10L 25/57

(57) ABSTRACT

A method automatically creating a highlight video from video data captured at a head-wearable device is described. The method includes, in response to a capture command, receiving image data captured at cameras of the head-wearable device and receiving audio data captured at microphones of the head-wearable device. The method further comprises causing the image data to be divided into a plurality of image segments, causing the audio data to be divided into a plurality of audio segments, each audio segment of the plurality of audio segments corresponding to a respective image segment of the plurality of image segments, and generating a respective transcription of each audio segment. The method 300 further includes, in accordance with a determination, based on the respective transcription, that a respective audio segment is a highlight segment, add the respective audio segment and a corresponding image segment to a highlight video.

### RELATED APPLICATION

This application claims priority to U.S. Provisional Application Serial No. 63/786,207, filed Apr. 9, 2025, entitled “Context-Aware Techniques For Video Highlight Extraction Integrating Audio Analysis, And System Of Use Thereof,” and U.S. Provisional Application Serial No. 63/795,115, filed Apr. 25, 2025, entitled “Context-Aware Techniques For Video Highlight Extraction Integrating Audio Analysis, And System Of Use Thereof,” which are incorporated herein by reference.

### TECHNICAL FIELD

This relates generally to video highlight extraction from video captured at a head-wearable device using audio and image analysis.

### BACKGROUND

Creating video highlights has traditionally been a challenging problem. Earlier approaches primarily relied on manually selected features extracted from video frames. These features included elements such as: (i) the presence of prominent or large faces, (ii) the appearance of pets, and (iii), specific frames that depicted smiles, laughter, and/or other visually engaging moments. While these handcrafted methods provide a basic framework for identifying potential highlights, they often resulted in suboptimal or mediocre outcomes. This is largely because they failed to capture nuanced and contextual aspects of what makes a moment genuinely significant or emotionally engaging. Additionally, these approaches are limited to visual data and do not fully leverage the rich multimodal information present in video data. With advancements in artificial intelligence, particularly with the emergence of large multimodal foundational models (such as large language models), the landscape has significantly evolved. These models enable the extraction of complex features from both image frames and audio in videos, offering a much deeper understanding of the content and context. Leveraging the zero-shot capabilities of these models can allow for creating video highlights in a more automated, context-aware, and scalable manner. Features of multi-modal artificial intelligence models such as Automatic Speech Recognition (ASR), generated image descriptions, and audio sentiment analysis may be utilized to identify key moments in videos to be automatically highlights.

As such, there is a need to address one or more of the above-identified challenges. A brief summary of solutions to the issues noted above are described below.

### SUMMARY

One example of a method for automatically creating a highlight video from video data captured at a head-wearable device is described herein. This example method occurs at a head-wearable device including one or more cameras and one or more microphones. The head-wearable device and/or another device communicatively coupled to the head-wearable device includes and one or more programs, where the one or more programs are stored in memory and configured to be executed by one or more processors. The one or more programs including instructions for performing operations. The operations include, in response to a capture command (e.g., a voice command, a hand gesture, and/or a touch input): (i) receiving image data captured at one or more cameras of the head-wearable device and (ii) receiving audio data captured at one or more microphones of the head-wearable device (e.g., the image data and the audio data together comprise video data captured at the head-wearable device). The operations further include: (i) causing the image data to be divided into a plurality of image segments, (ii) causing the audio data to be divided into a plurality of audio segments, each audio segment of the plurality of audio segments corresponding to a respective image segment of the plurality of image segments (e.g., each respective audio segment and each corresponding image segment comprise a video data segment), and (iii) generating a respective transcription of each audio segment of the plurality of audio segments. The operations further include, in accordance with a determination, based on the respective transcription, that a respective audio segment is a highlight segment, add the respective audio segment and a corresponding image segment (e.g., the respective audio segment and the corresponding image segment comprise a video segment) to a highlight video. In some embodiments, the highlight video is then presented to a user of the head-wearable device and/or is stored at a memory device associated with the user (e.g., a hard drive of a device of the user and/or a cloud server associated with the user).

Instructions that cause performance of the methods and operations described herein can be stored on a non-transitory computer readable storage medium. The non-transitory computer-readable storage medium can be included on a single electronic device or spread across multiple electronic devices of a system (computing system). A non-exhaustive of list of electronic devices that can either alone or in combination (e.g., a system) perform the method and operations described herein include an extended-reality (XR) headset/glasses (e.g., a mixed-reality (MR) headset or a pair of augmented-reality (AR) glasses as two examples), a wrist-wearable device, an intermediary processing device, a smart textile-based garment, etc. For instance, the instructions can be stored on a pair of AR glasses or can be stored on a combination of a pair of AR glasses and an associated input device (e.g., a wrist-wearable device) such that instructions for causing detection of input operations can be performed at the input device and instructions for causing changes to a displayed user interface in response to those input operations can be performed at the pair of AR glasses. The devices and systems described herein can be configured to be used in conjunction with methods and operations for providing an XR experience. The methods and operations for providing an XR experience can be stored on a non-transitory computer-readable storage medium.

The features and advantages described in the specification are not necessarily all inclusive and, in particular, certain additional features and advantages will be apparent to one of ordinary skill in the art in view of the drawings, specification, and claims. Moreover, it should be noted that the language used in the specification has been principally selected for readability and instructional purposes.

Having summarized the above example aspects, a brief description of the drawings will now be presented.

### BRIEF DESCRIPTION OF THE DRAWINGS

For a better understanding of the various described embodiments, reference should be made to the Detailed Description below, in conjunction with the following drawings in which like reference numerals refer to corresponding parts throughout the figures.

FIG. 1A illustrates an example of a user interacting with a video highlight auto-capture feature at a head-wearable device, in accordance with some embodiments.

FIG. 1B illustrates a sequence of video segments captured while a user is on a hike, in accordance with some embodiments.

FIG. 2 illustrates an example diagram of a video highlight auto-capture feature automatically generating a highlight video from video data captured at a head-wearable device, in accordance with some embodiments.

FIG. 3 illustrates a flow diagram of a method of automatically creating a highlight video from video data captured at a head-wearable device, in accordance with some embodiments.

FIGS. 4A, 4B, 4C-1 and 4C-2, illustrate example MR and AR systems, in accordance with some embodiments.

In accordance with common practice, the various features illustrated in the drawings may not be drawn to scale. Accordingly, the dimensions of the various features may be arbitrarily expanded or reduced for clarity. In addition, some of the drawings may not depict all of the components of a given system, method, or device. Finally, like reference numerals may be used to denote like features throughout the specification and figures.

### DETAILED DESCRIPTION

Numerous details are described herein to provide a thorough understanding of the example embodiments illustrated in the accompanying drawings. However, some embodiments may be practiced without many of the specific details, and the scope of the claims is only limited by those features and aspects specifically recited in the claims. Furthermore, well-known processes, components, and materials have not necessarily been described in exhaustive detail so as to avoid obscuring pertinent aspects of the embodiments described herein.

### Overview

Embodiments of this disclosure can include or be implemented in conjunction with various types of extended-realities (XRs) such as mixed-reality (MR) and augmented-reality (AR) systems. MRs and ARs, as described herein, are any superimposed functionality and/or sensory-detectable presentation provided by MR and AR systems within a user’s physical surroundings. Such MRs can include and/or represent virtual realities (VRs) and VRs in which at least some aspects of the surrounding environment are reconstructed within the virtual environment (e.g., displaying virtual reconstructions of physical objects in a physical environment to avoid the user colliding with the physical objects in a surrounding physical environment). In the case of MRs, the surrounding environment that is presented through a display is captured via one or more sensors configured to capture the surrounding environment (e.g., a camera sensor, time-of-flight (ToF) sensor). While a wearer of an MR headset can see the surrounding environment in full detail, they are seeing a reconstruction of the environment reproduced using data from the one or more sensors (i.e., the physical objects are not directly viewed by the user). An MR headset can also forgo displaying reconstructions of objects in the physical environment, thereby providing a user with an entirely VR experience. An AR system, on the other hand, provides an experience in which information is provided, e.g., through the use of a waveguide, in conjunction with the direct viewing of at least some of the surrounding environment through a transparent or semi-transparent waveguide(s) and/or lens(es) of the AR glasses. Throughout this application, the term “extended reality (XR)” is used as a catchall term to cover both ARs and MRs. In addition, this application also uses, at times, a head-wearable device or headset device as a catchall term that covers XR headsets such as AR glasses and MR headsets.

As alluded to above, an MR environment, as described herein, can include, but is not limited to, non-immersive, semi-immersive, and fully immersive VR environments. As also alluded to above, AR environments can include marker-based AR environments, markerless AR environments, location-based AR environments, and projection-based AR environments. The above descriptions are not exhaustive and any other environment that allows for intentional environmental lighting to pass through to the user would fall within the scope of an AR, and any other environment that does not allow for intentional environmental lighting to pass through to the user would fall within the scope of an MR.

The AR and MR content can include video, audio, haptic events, sensory events, or some combination thereof, any of which can be presented in a single channel or in multiple channels (such as stereo video that produces a three-dimensional effect to a viewer). Additionally, AR and MR can also be associated with applications, products, accessories, services, or some combination thereof, which are used, for example, to create content in an AR or MR environment and/or are otherwise used in (e.g., to perform activities in) AR and MR environments.

Interacting with these AR and MR environments described herein can occur using multiple different modalities and the resulting outputs can also occur across multiple different modalities. In one example AR or MR system, a user can perform a swiping in-air hand gesture to cause a song to be skipped by a song-providing application programming interface (API) providing playback at, for example, a home speaker.

A hand gesture, as described herein, can include an in-air gesture, a surface-contact gesture, and or other gestures that can be detected and determined based on movements of a single hand (e.g., a one-handed gesture performed with a user’s hand that is detected by one or more sensors of a wearable device (e.g., electromyography (EMG) and/or inertial measurement units (IMUs) of a wrist-wearable device, and/or one or more sensors included in a smart textile wearable device) and/or detected via image data captured by an imaging device of a wearable device (e.g., a camera of a head-wearable device, an external tracking camera setup in the surrounding environment)). “In-air” generally includes gestures in which the user’s hand does not contact a surface, object, or portion of an electronic device (e.g., a head-wearable device or other communicatively coupled device, such as the wrist-wearable device), in other words the gesture is performed in open air in 3D space and without contacting a surface, an object, or an electronic device. Surface-contact gestures (contacts at a surface, object, body part of the user, or electronic device) more generally are also contemplated in which a contact (or an intention to contact) is detected at a surface (e.g., a single- or double-finger tap on a table, on a user’s hand or another finger, on the user’s leg, a couch, a steering wheel). The different hand gestures disclosed herein can be detected using image data and/or sensor data (e.g., neuromuscular signals sensed by one or more biopotential sensors (e.g., EMG sensors) or other types of data from other sensors, such as proximity sensors, ToF sensors, sensors of an IMU, capacitive sensors, strain sensors) detected by a wearable device worn by the user and/or other electronic devices in the user’s possession (e.g., smartphones, laptops, imaging devices, intermediary devices, and/or other devices described herein).

A gaze gesture, as described herein, can include an eye movement and/or a head movement indicative of a location of a gaze of the user, an implied location of the gaze of the user, and/or an approximated location of the gaze of the user, in the surrounding environment, the virtual environment, and/or the displayed user interface. The gaze gesture can be detected and determined based on (i) eye movements captured by one or more eye-tracking cameras (e.g., one or more cameras positioned to capture image data of one or both eyes of the user) and/or (ii) a combination of a head orientation of the user (e.g., based on head and/or body movements) and image data from a point-of-view camera (e.g., a forward-facing camera of the head-wearable device). The head orientation is determined based on IMU data captured by an IMU sensor of the head-wearable device. In some embodiments, the IMU data indicates a pitch angle (e.g., the user nodding their head up-and-down) and a yaw angle (e.g., the user shaking their head side-to-side). The head-orientation can then be mapped onto the image data captured from the point-of-view camera to determine the gaze gesture. For example, a quadrant of the image data that the user is looking at can be determined based on whether the pitch angle and the yaw angle are negative or positive (e.g., a positive pitch angle and a positive yaw angle indicate that the gaze gesture is directed toward a top-left quadrant of the image data, a negative pitch angle and a negative yaw angle indicate that the gaze gesture is directed toward a bottom-right quadrant of the image data, etc.). In some embodiments, the IMU data and the image data used to determine the gaze are captured at a same time, and/or the IMU data and the image data used to determine the gaze are captured at offset times (e.g., the IMU data is captured at a predetermined time (e.g., 0.01 seconds to 0.5 seconds) after the image data is captured). In some embodiments, the head-wearable device includes a hardware clock to synchronize the capture of the IMU data and the image data. In some embodiments, object segmentation and/or image detection methods are applied to the quadrant of the image data that the user is looking at.

The input modalities as alluded to above can be varied and are dependent on a user’s experience. For example, in an interaction in which a wrist-wearable device is used, a user can provide inputs using in-air or surface-contact gestures that are detected using neuromuscular signal sensors of the wrist-wearable device. In the event that a wrist-wearable device is not used, alternative and entirely interchangeable input modalities can be used instead, such as camera(s) located on the headset/glasses or elsewhere to detect in-air or surface-contact gestures or inputs at an intermediary processing device (e.g., through physical input components (e.g., buttons and trackpads)). These different input modalities can be interchanged based on both desired user experiences, portability, and/or a feature set of the product (e.g., a low-cost product may not include hand-tracking cameras).

While the inputs are varied, the resulting outputs stemming from the inputs are also varied. For example, an in-air gesture input detected by a camera of a head-wearable device can cause an output to occur at a head-wearable device or control another electronic device different from the head-wearable device. In another example, an input detected using data from a neuromuscular signal sensor can also cause an output to occur at a head-wearable device or control another electronic device different from the head-wearable device. While only a couple examples are described above, one skilled in the art would understand that different input modalities are interchangeable along with different output modalities in response to the inputs.

Specific operations described above may occur as a result of specific hardware. The devices described are not limiting and features on these devices can be removed or additional features can be added to these devices. The different devices can include one or more analogous hardware components. For brevity, analogous devices and components are described herein. Any differences in the devices and components are described below in their respective sections.

As described herein, a processor (e.g., a central processing unit (CPU) or microcontroller unit (MCU)), is an electronic component that is responsible for executing instructions and controlling the operation of an electronic device (e.g., a wrist-wearable device, a head-wearable device, a handheld intermediary processing device (HIPD), a smart textile-based garment, or other computer system). There are various types of processors that may be used interchangeably or specifically required by embodiments described herein. For example, a processor may be (i) a general processor designed to perform a wide range of tasks, such as running software applications, managing operating systems, and performing arithmetic and logical operations; (ii) a microcontroller designed for specific tasks such as controlling electronic devices, sensors, and motors; (iii) a graphics processing unit (GPU) designed to accelerate the creation and rendering of images, videos, and animations (e.g., VR animations, such as three-dimensional modeling); (iv) a field-programmable gate array (FPGA) that can be programmed and reconfigured after manufacturing and/or customized to perform specific tasks, such as signal processing, cryptography, and machine learning; or (v) a digital signal processor (DSP) designed to perform mathematical operations on signals such as audio, video, and radio waves. One of skill in the art will understand that one or more processors of one or more electronic devices may be used in various embodiments described herein.

As described herein, controllers are electronic components that manage and coordinate the operation of other components within an electronic device (e.g., controlling inputs, processing data, and/or generating outputs). Examples of controllers can include (i) microcontrollers, including small, low-power controllers that are commonly used in embedded systems and Internet of Things (IoT) devices; (ii) programmable logic controllers (PLCs) that may be configured to be used in industrial automation systems to control and monitor manufacturing processes; (iii) system-on-a-chip (SoC) controllers that integrate multiple components such as processors, memory, I/O interfaces, and other peripherals into a single chip; and/or (iv) DSPs. As described herein, a graphics module is a component or software module that is designed to handle graphical operations and/or processes and can include a hardware module and/or a software module.

As described herein, memory refers to electronic components in a computer or electronic device that store data and instructions for the processor to access and manipulate. The devices described herein can include volatile and non-volatile memory. Examples of memory can include (i) random access memory (RAM), such as DRAM, SRAM, DDR RAM or other random access solid state memory devices, configured to store data and instructions temporarily; (ii) read-only memory (ROM) configured to store data and instructions permanently (e.g., one or more portions of system firmware and/or boot loaders); (iii) flash memory, magnetic disk storage devices, optical disk storage devices, other non-volatile solid state storage devices, which can be configured to store data in electronic devices (e.g., universal serial bus (USB) drives, memory cards, and/or solid-state drives (SSDs)); and (iv) cache memory configured to temporarily store frequently accessed data and instructions. Memory, as described herein, can include structured data (e.g., SQL databases, MongoDB databases, GraphQL data, or JSON data). Other examples of memory can include (i) profile data, including user account data, user settings, and/or other user data stored by the user; (ii) sensor data detected and/or otherwise obtained by one or more sensors; (iii) media content data including stored image data, audio data, documents, and the like; (iv) application data, which can include data collected and/or otherwise obtained and stored during use of an application; and/or (v) any other types of data described herein.

As described herein, a power system of an electronic device is configured to convert incoming electrical power into a form that can be used to operate the device. A power system can include various components, including (i) a power source, which can be an alternating current (AC) adapter or a direct current (DC) adapter power supply; (ii) a charger input that can be configured to use a wired and/or wireless connection (which may be part of a peripheral interface, such as a USB, micro-USB interface, near-field magnetic coupling, magnetic inductive and magnetic resonance charging, and/or radio frequency (RF) charging); (iii) a power-management integrated circuit, configured to distribute power to various components of the device and ensure that the device operates within safe limits (e.g., regulating voltage, controlling current flow, and/or managing heat dissipation); and/or (iv) a battery configured to store power to provide usable power to components of one or more electronic devices.

As described herein, peripheral interfaces are electronic components (e.g., of electronic devices) that allow electronic devices to communicate with other devices or peripherals and can provide a means for input and output of data and signals. Examples of peripheral interfaces can include (i) USB and/or micro-USB interfaces configured for connecting devices to an electronic device; (ii) Bluetooth interfaces configured to allow devices to communicate with each other, including Bluetooth low energy (BLE); (iii) near-field communication (NFC) interfaces configured to be short-range wireless interfaces for operations such as access control; (iv) pogo pins, which may be small, spring-loaded pins configured to provide a charging interface; (v) wireless charging interfaces; (vi) global-positioning system (GPS) interfaces; (vii) Wi-Fi interfaces for providing a connection between a device and a wireless network; and (viii) sensor interfaces.

As described herein, sensors are electronic components (e.g., in and/or otherwise in electronic communication with electronic devices, such as wearable devices) configured to detect physical and environmental changes and generate electrical signals. Examples of sensors can include (i) imaging sensors for collecting imaging data (e.g., including one or more cameras disposed on a respective electronic device, such as a simultaneous localization and mapping (SLAM) camera); (ii) biopotential-signal sensors; (iii) IMUs for detecting, for example, angular rate, force, magnetic field, and/or changes in acceleration; (iv) heart rate sensors for measuring a user’s heart rate; (v) peripheral oxygen saturation (SpO₂) sensors for measuring blood oxygen saturation and/or other biometric data of a user; (vi) capacitive sensors for detecting changes in potential at a portion of a user’s body (e.g., a sensor-skin interface) and/or the proximity of other devices or objects; (vii) sensors for detecting some inputs (e.g., capacitive and force sensors); and (viii) light sensors (e.g., ToF sensors, infrared light sensors, or visible light sensors), and/or sensors for sensing data from the user or the user’s environment. As described herein biopotential-signal-sensing components are devices used to measure electrical activity within the body (e.g., biopotential-signal sensors). Some types of biopotential-signal sensors include (i) electroencephalography (EEG) sensors configured to measure electrical activity in the brain to diagnose neurological disorders; (ii) electrocardiography (ECG or EKG) sensors configured to measure electrical activity of the heart to diagnose heart problems; (iii) EMG sensors configured to measure the electrical activity of muscles and diagnose neuromuscular disorders; (iv) electrooculography (EOG) sensors configured to measure the electrical activity of eye muscles to detect eye movement and diagnose eye disorders.

As described herein, an application stored in memory of an electronic device (e.g., software) includes instructions stored in the memory. Examples of such applications include (i) games; (ii) word processors; (iii) messaging applications; (iv) media-streaming applications; (v) financial applications; (vi) calendars; (vii) clocks; (viii) web browsers; (ix) social media applications; (x) camera applications; (xi) web-based applications; (xii) health applications; (xiii) AR and MR applications; and/or (xiv) any other applications that can be stored in memory. The applications can operate in conjunction with data and/or one or more components of a device or communicatively coupled devices to perform one or more operations and/or functions.

As described herein, communication interface modules can include hardware and/or software capable of data communications using any of a variety of custom or standard wireless protocols (e.g., IEEE 802.15.4, Wi-Fi, ZigBee, 6LoWPAN, Thread, Z-Wave, Bluetooth Smart, ISA100.11a, WirelessHART, or MiWi), custom or standard wired protocols (e.g., Ethernet or HomePlug), and/or any other suitable communication protocol, including communication protocols not yet developed as of the filing date of this document. A communication interface is a mechanism that enables different systems or devices to exchange information and data with each other, including hardware, software, or a combination of both hardware and software. For example, a communication interface can refer to a physical connector and/or port on a device that enables communication with other devices (e.g., USB, Ethernet, HDMI, or Bluetooth). A communication interface can refer to a software layer that enables different software programs to communicate with each other (e.g., APIs and protocols such as HTTP and TCP/IP).

As described herein, a graphics module is a component or software module that is designed to handle graphical operations and/or processes and can include a hardware module and/or a software module.

As described herein, non-transitory computer-readable storage media are physical devices or storage medium that can be used to store electronic data in a non-transitory form (e.g., such that the data is stored permanently until it is intentionally deleted and/or modified).

### Augmenting Automatic Highlight Extraction With Audio Analysis

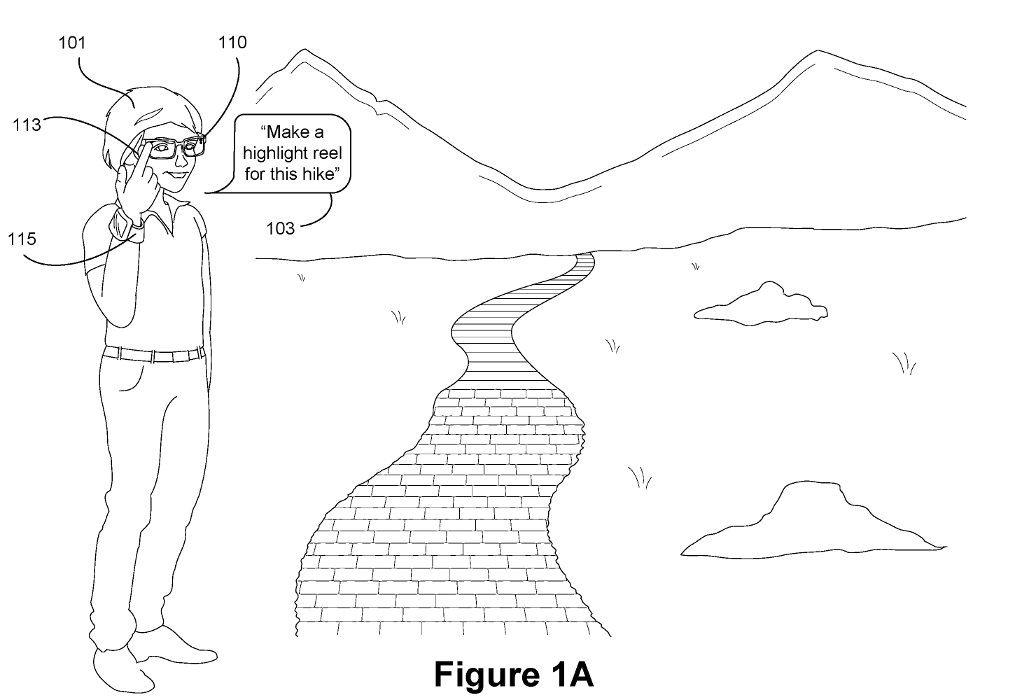

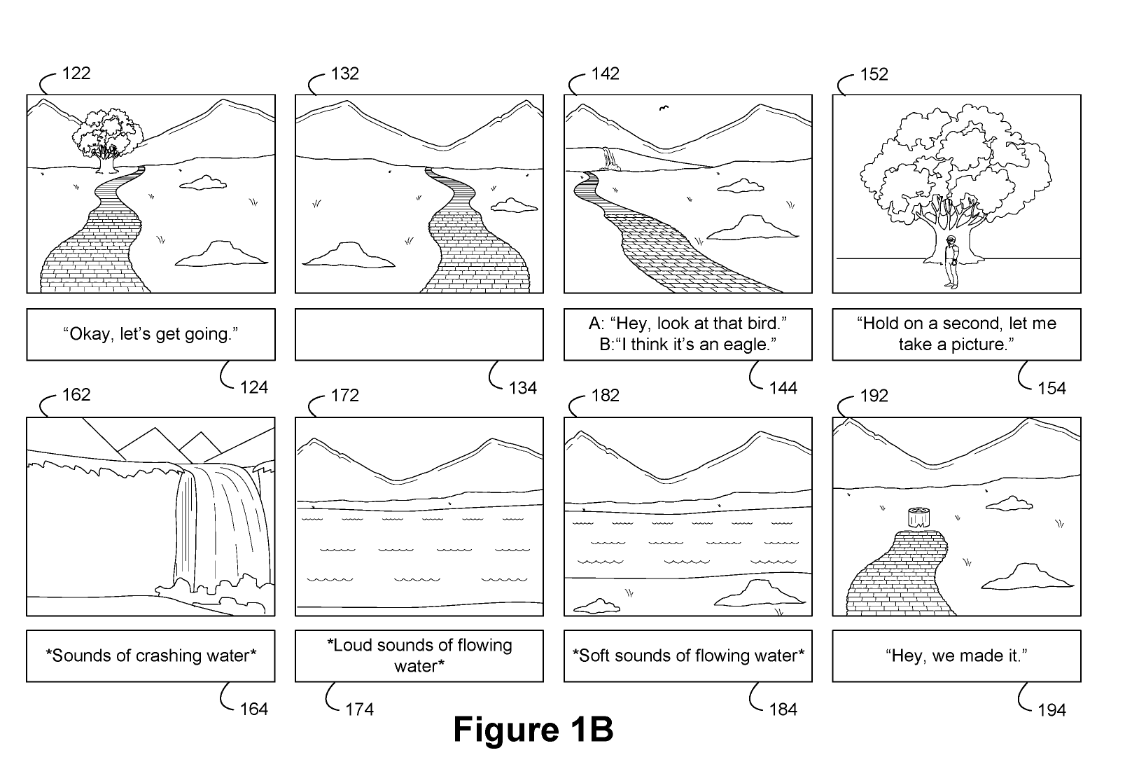

FIG. 1A illustrates an example of a user 101 interacting with a video highlight auto-capture feature at a head-wearable device 110, in accordance with some embodiments. The video highlight auto-capture feature is executed at one or more processors of the head-wearable device 110 (e.g., a pair of smart glasses, an extended-reality headset, smart contacts, a head-mounted camera, and/or a smart hat), another device associated with the user 101 and communicatively coupled to the head-wearable device 110 (e.g., a smart watch 115, a smartphone, a personal computer, a handheld intermediary processing device, etc.), and/or a server device communicatively coupled to the head-wearable device 110 and/or the other device (e.g., the other device acts an intermediate device to communicate data between the head-wearable device and the server device). The head-wearable device 110 includes one or more imaging devices (e.g., forward-facing cameras) configured to capture image data and one or more audio devices (e.g., microphones) configured to capture audio data. In some embodiments, the other device includes one or more other imaging devices and one or more other audio devices that capture other image data and other audio data, respectively. In some embodiments, the other image data and/or the other audio data may be substituted for and/or supplement the image data and/or the audio data. The head-wearable device 110 and/or the other device further includes one or more processors and one or more computer-readable storage media including executable instructions that, when executed by one or more processors, cause the one or more processors to execute tasks related to the video highlight auto-capture feature. In some embodiments, a first portion of the tasks related to the video highlight auto-capture feature are executed at one or more processors of the head-wearable device 110, and a second portion of the tasks related to the video highlight auto-capture feature are executed at one or more processors of the other device.

The video highlight auto-capture feature includes capturing the image data and the audio data (the image data and the audio data together are video data) at the head-wearable device 110 over a period-of-time (e.g., thirty minutes). Once the period-of-time has passed, the video highlight auto-capture feature selects a plurality of highlight segments from the video data and compiles the plurality of highlight segments into a highlight video (e.g., one minute in length). In some embodiments, a remainder of segments from the video data that are not selected as highlight segments are discarded (e.g., deleted). The highlight video is then presented to the user 101 (e.g., at one or more displays and/or one or more speakers of the head-wearable device 110, the other device, and/or an additional device communicatively coupled to the head-wearable device 110 and/or the other device) and/or is stored in a computer-readable storage device associated with the user 101 (e.g., a computer-readable memory of the head-wearable device 110, a computer-readable memory of the other device, and/or a cloud memory storage device associated with the user 101). In some embodiments, the video highlight auto-capture feature uses a multi-modal artificial intelligence (AI) (e.g., the multi-modal AI includes automatic speech recognition (ASR), computer vision, a language-learning model, etc.) to determine which segments of the video data are highlight segments (e.g., segments which are more exciting, have interesting items/objects/actions/events/people/conversations in them, etc.). The multi-modal AI determines which segments of the video data are highlight segments based on the image data and/or the audio data. For example, a particular object or a particular subject 105 in the image data may indicate, to the multi-modal AI, that the segment of video should be highlighted, and/or a person saying a key word or key phrase (or cheering, loud noises, non-verbal sounds, etc.) in the audio data may indicate, to the multi-modal AI, that the segment of video should be highlighted. In some embodiments, the video highlight auto-capture feature is initiated in response to a capture command. The capture command is one or more of a voice command 103 (e.g., “Make a highlight reel of this hike.”), a touch input 113 (e.g., the user 101 touches a side of the head-wearable device 110), and/or a hand gesture (e.g., a finger-snap gesture). In some embodiments, the capture command includes one or more context clues that the multi-modal AI utilizes to determine which segments of the video data are highlight segments. For example, if the capture command is “Make a highlight reel of this hike,” the multi-modal AI will select segments of the video data corresponding to hiking (e.g., viewpoints, waterfalls, a person saying “Wow, look at that,” etc.). As another example, if the capture command is “Make a highlight reel of this basketball game,” the multi-modal AI will select segments of the video data corresponding to basketball (e.g., scores, steals, moments when a crowd cheers, etc.).

In some embodiments, the period-of-time is a preset period-of-time, a period-of-time based on one or more user settings, and/or a period-of-time determined by the multi-modal AI (e.g., based on the capture command, the video data, a battery level of the head-wearable device 110, a one or more context clues). In some embodiments, after the video data has been captured over the period-of-time, it is divided into a plurality of segments. Each segment of the plurality of segments are a predetermined length between five seconds and thirty seconds (e.g., sixteen second segments). In some embodiments, the multi-modal AI divides the video data into the plurality of segments based on the image data and/or the audio data. For example, the multi-modal AI determines a point in the video data to divide two video segments based on a change in identifiable items/objects/actions/events/people in the image data and/or a pause in speech in the audio data.

The multi-modal AI determines whether each segment is a highlight segment based on respective image data of each segment (an image segment) and/or respective audio data of each segment (an audio segment). In some embodiments, the multi-modal AI determines whether each segment is a highlight segment based on a transcription of the respective audio segment. In response to capturing the audio data, each audio segment of the audio data is transcribed by ASR into a respective segment transcription. In some embodiments, the multi-modal AI determines whether each respective segment is a highlight segment in accordance with a determination that the respective transcription includes one or more key words and/or key phrases (e.g., “Cool” and/or “Woah, look at that”). In some embodiments, the one or more key words and/or key phrases are based on the one or more context clues included in the capture command. For example, if the capture command is “Make a highlight reel of this basketball game,” the one or more key words and/or key phrases may include cheering, “score,” a buzzer noise, etc.

In some embodiments, the multi-modal AI determines whether each segment is a highlight segment based on one or more emotion/sentiment tags of the respective audio segment. As an example, the multi-modal AI assigns a score (e.g., a percentage of likelihood) for a plurality of emotion/sentiment categories (e.g., angry, calm, disgusted, happy, etc.) to each segment, and selects one or more of the emotion/sentiment categories to tag each segment. The multi-modal AI selects one or more of the emotion/sentiment tags based on whether a respective score of the one or more of emotion/sentiment categories exceeds an emotion/sentiment category threshold (e.g., 20% likelihood), and/or the multi-modal AI selects one or more of the emotion/sentiment tags which of the one or more of emotion/sentiment has a greatest respective score. The multi-modal AI then determines whether the respective segment is a highlight segment based whether the respective audio segment has one or more desirable emotion/sentiment tags (e.g., happy, calm excited, etc.) and/or whether the respective audio segment has a desirable score for one or more of the plurality of emotion/sentiment categories. In some embodiments, the one or more desirable emotion/sentiment tags and/or the desirable score for the one or more of the plurality of emotion/sentiment categories is based on the capture command. For example, if the capture command is “Make a highlight reel of this bike ride,” the desirable emotion/sentiment tags may include happy, calm, playful, etc.

In some embodiments, the multi-modal AI determines whether each segment is a highlight segment based on an amount of background noise (e.g., people talking, traffic, wind, etc.) in the respective audio segment. In response to capturing the audio data, the multi-modal AI determines (e.g., via ASR) a proportion of the respective audio segment that is background noise (e.g., as a percentage of the audio segment). In some embodiments, the multi-modal AI determines that a respective segment is a highlight segment if the background noise is below a background noise threshold (e.g., less than 20% of the respective audio segment), and/or the multi-modal AI determines that the respective segment is not a highlight segment if the background noise is above the background noise threshold.

In some embodiments, the multi-modal AI determines whether each segment is a highlight segment based on an amount of a number of speakers in the respective audio segment. In response to capturing the audio data, the multi-modal AI determines (e.g., via ASR) a number of speakers in the respective audio segment. In some embodiments, the multi-modal AI determines that a respective segment is a highlight segment if the number of speakers is above a speaker threshold (e.g., two or more speakers), and/or the multi-modal AI determines that the respective segment is a highlight segment if the speaker is below the speaker threshold. In some embodiments, a value of the speaker threshold and/or whether the speaker threshold is satisfied by the number of speakers being greater or less than the speaker threshold, are based on the one or more context clues included in the capture command. For example, the multi-modal AI may determine that the respective segment is a highlight segment if the number of speakers is above the speaker threshold when the capture command is “Make a highlight reel of this party.” The multi-modal AI may determine that the respective segment is a highlight segment if the number of speakers is below the speaker threshold when the capture command is “Make a highlight reel of this singing recital.”

In some embodiments, the multi-modal AI determines whether each segment is a highlight segment based on an amount of a number of spoken words in the respective audio segment. In response to capturing the audio data, the multi-modal AI determines (e.g., via ASR) a number of spoken words in the respective audio segment. In some embodiments, the multi-modal AI determines that a respective segment is a highlight segment if the number of spoken words is above a word threshold (e.g., ten or more words), and/or the multi-modal AI determines that the respective segment is a highlight segment if the speaker is below the word threshold. In some embodiments, a value of the word threshold and/or whether the word threshold is satisfied by the number of spoken words being greater or less than the word threshold, are based on the one or more context clues included in the capture command. For example, the multi-modal AI may determine that the respective segment is a highlight segment if the number of spoken words is above the word threshold when the capture command is “Make a highlight reel of this speech.” The multi-modal AI may determine that the respective segment is a highlight segment if the number of spoken words is below the word threshold when the capture command is “Make a highlight reel of this fishing trip.”

In some embodiments, the multi-modal AI determines whether each segment is a highlight segment based on one or more objects of interest in the respective image segment. In response to capturing the image data, the multi-modal AI determines (e.g., via computer vision) one or more objects (including items, people, animals, landscapes, buildings, etc.) in the respective image segment. In some embodiments, the multi-modal AI determines that a respective segment is a highlight segment if the one or more objects includes one or more objects of interest (e.g., two or more speakers). In some embodiments, the one or more objects of interest are based on the one or more context clues included in the capture command. In some embodiments, the multi-modal AI generates, based on the image segment, a caption of the segment (e.g., a textual description of the image segment), and the multi-modal AI then determines whether each segment is a highlight segment based on the caption of the segment. In some embodiments, the caption of the segment describes the one or more objects in the respective image segment.

In some embodiments, the multi-modal AI determines whether each segment is a highlight segment based on a health signal (e.g., heart rate, pupil dilation, breathing rate, etc.) of the user 101. While capturing the image data and the audio data, a health signal sensor (e.g., a body microphone, an eye-tracking device, an IMU sensor, etc.) of the head-wearable device 110 and/or the other device captures the health signal from the user 101 over the period of time. The health sensor signal includes a plurality of health signal segments, each health signal segment of the plurality of health signal segments corresponding to a respective segment. In some embodiments, the multi-modal AI determines that a respective segment is a highlight segment if a respective health signal segment indicates user excitement. For example, an elevated heart rate, dilated pupils, and/or an elevated breathing rate indicate that the user 101 is excited. In some embodiments, the multi-modal AI determines that a respective segment is a highlight segment if a respective health signal segment indicates user relaxation. For example, a lowered heart rate and/or a lowered breathing rate indicate that the user 101 is relaxed during an auto-capture session wherein the user 101 is active (e.g., the user 101 stops to look at objects/places while on a run). In some embodiments, the multi-modal AI determines that a respective segment is a highlight segment if the respective health signal segment is above a health signal threshold (e.g., a heart rate above 110 beats per minutes, a pupil dilation of 25 percent, a breathing rate above 25 breaths per minute), and/or the multi-modal AI determines that the respective segment is a highlight segment if the respective health signal segment is below the health signal threshold. In some embodiments, one or more emotion/sentiment tags of the respective audio segment are further based on the respective health signal segment.

In some embodiments, the multi-modal AI determines whether each segment is a highlight segment based on a gaze of the user 101. While capturing the image data and the audio data, a gaze-tracking device (e.g., an eye-tracking camera and/or an IMU sensor providing IMU data that is used to approximate gaze location) of the head-wearable device 110 the gaze of the user 101 over the period of time. The gaze of the user 101 includes a plurality of gaze segments, each gaze segment of the plurality of gaze segments corresponding to a respective segment. In some embodiments, the multi-modal AI determines that a respective segment is a highlight segment if a respective gaze segment indicates user excitement. For example, the gaze of the user 101 frequently moving in the respective gaze segment indicate that the user 101 is excited. In some embodiments, the multi-modal AI determines that a respective segment is a highlight segment if a respective gaze segment indicates that the user 101 is focused on an object. For example, the gaze of the user 101 remaining in a single location indicates that the user 101 is focused on an object.

In some embodiments, the multi-modal AI determines whether each segment is a highlight segment based on a combination of the methods listed above. For example, the multi-modal AI assigns multiple highlight factors for each of the methods listed above for each segment, and, based on a total highlight factor, determines whether each segment is a highlight segment (e.g., if the total highlight factor exceeds a factor threshold). In some embodiments, the multi-modal AI assigns a keyword highlight factor to the respective segment based on whether the respective transcription contains the one or more key words and/or key phrases, and/or assigns different keyword highlight factors based on a type of key word and/or key phrase. For example, a keyword highlight factor of 0.0 is assigned when no key words or key phrases are in the respective transcription, a keyword highlight factor of 0.5 is assigned when the key word “Cool” is in the respective transcription, and a keyword highlight factor of 1.0 is assigned when the key phrase “That’s awesome” is in the respective transcription. In some embodiments, the multi-modal AI assigns an emotion/sentiment highlight factor to the respective segment based on the emotion/sentiment tag, and/or assigns different emotion/sentiment highlight factors based on the scores of the one or more of emotion/sentiment categories. For example, an emotion/sentiment highlight factor of 0.0 is assigned when the emotion/sentiment tag is a “sad” tag, an emotion/sentiment highlight factor of 0.5 is assigned when the emotion/sentiment is a “calm” tag, and an emotion/sentiment highlight factor of 1.0 is assigned when the emotion/sentiment tag is an “excited” tag. In some embodiments, the multi-modal AI assigns a background noise highlight factor to the respective segment based on the proportion of the respective audio segment that is background noise, and/or assigns different emotion/sentiment highlight factors based on whether the background noise is above or below the background noise threshold. In some embodiments, the multi-modal AI assigns a speaker highlight factor to the respective segment based on the number of speakers in the respective audio segment, and/or assigns different speaker highlight factors based on whether the number of speakers is above or below the speaker threshold. In some embodiments, the multi-modal AI assigns an object highlight factor to the respective segment based on a number of the one or more objects of interest in the image segment, and/or a type of the one or more objects of interest in the image segment. In some embodiments, the multi-modal AI assigns a health signal highlight factor (e.g., a heart rate factor, a pupil dilation factor, a breathing rate factor, etc.) to the respective segment based on the health signal in a corresponding health signal segment, and/or assigns different health signal highlight factors based on whether the heath signal is above or below the health signal threshold. In some embodiments, the multi-modal AI assigns a gaze highlight factor to the respective segment based on the gaze of the user 101 in a corresponding gaze segment.

FIG. 1B illustrates a sequence of video segments captured while the user 101 is on a hike, in accordance with some embodiments. Each of the eight video segments illustrated in FIG. 1B comprises a respective image segment and a respective audio segment. In some embodiments, the video highlight auto-capture feature begins capturing the video data in response to the capture command “Captures some highlights from this hike.” A first video segment includes a first image segment 122 showing the user 101 beginning the hike, and a first audio segment 124 showing the user 101 saying “Okay, let’s get going.” In accordance with some embodiments, the multi-modal AI determines that the first video segment is a highlight segment based on a determination that the first video segment is the user 101 beginning the event that was requested in the capture command. A second video segment includes a second image segment 132 showing the user 101 continuing the hike, and a second audio segment 134 showing the user 101 saying nothing. In accordance with some embodiments, the multi-modal AI determines that the second video segment is not a highlight segment based on a determination that that there is nothing worth highlighting in the second image segment 132 and/or the second audio segment 134. A third video segment includes a third image segment 142 showing the user 101 continuing the hike, and a third audio segment 144 showing the user 101 saying “Hey, look at that bird” and a companion of the user 101 saying “I think it’s an eagle.” In accordance with some embodiments, the multi-modal AI determines that the third video segment is a highlight segment based on a determination that that there are multiple speakers in the third audio segment 144 and/or a determination that there is an object of interest (e.g., an eagle) in the third image segment 142, based on the third audio segment 144 (e.g., the multi-modal AI may not be able to identify that the eagle is present in the third video segment based on the third image segment 142, but the multi-modal AI is able to determine that the eagle is in the third image segment 142 based on the third audio segment 144). A fourth video segment includes a fourth image segment 152 showing the user 101 looking at the companion front of a large tree, and a fourth audio segment 154 showing the user 101 saying “Hold on a second, let me take a picture.” In accordance with some embodiments, the multi-modal AI determines that the fourth video segment is a highlight segment based on a determination that that the fourth audio segment 154 includes a keyword (e.g., “picture”). A fifth video segment includes a fifth image segment 162 showing the user 101 looking at a waterfall, and a fifth audio segment 164 showing waterfall sounds. In accordance with some embodiments, the multi-modal AI determines that the fifth video segment is a highlight segment based on a determination that that the fifth image segment 162 includes an object of interest (e.g., the waterfall). In some embodiments, the object of interest is based on the capture command (e.g., since the user 101 requested highlights from a hike, a waterfall is an object of interest). A sixth video segment includes a sixth image segment 172 showing the user 101 looking at a lake, and a sixth audio segment 174 showing sounds of a loud flowing river. In accordance with some embodiments, the multi-modal AI determines that the sixth video segment is not a highlight segment based on a determination that that the sixth audio segment 174 includes background noise above a background noise threshold (e.g., 80 decibels). A seventh video segment includes a seventh image segment 182 showing the user 101 looking at the lake from a different viewpoint from the sixth image segment 182, and a seventh audio segment 184 showing sounds of a softer flowing river. In accordance with some embodiments, the multi-modal AI determines that the seventh video segment is a highlight segment based on a determination that that the seventh audio segment 184 includes background noise below the background noise threshold and a determination that that the seventh image segment 182 includes another object of interest (e.g., the lake). An eighth video segment includes an eighth image segment 192 showing the user 101 ending the hike and an eighth audio segment 194 showing the user 101 saying “Hey, we made it.” In accordance with some embodiments, the multi-modal AI determines that the eighth video segment is a highlight segment based on a determination that that the eighth audio segment 194 includes background noise below the background noise threshold and a determination that that the eighth video segment is the user 101 ending the event that was requested in the capture command.

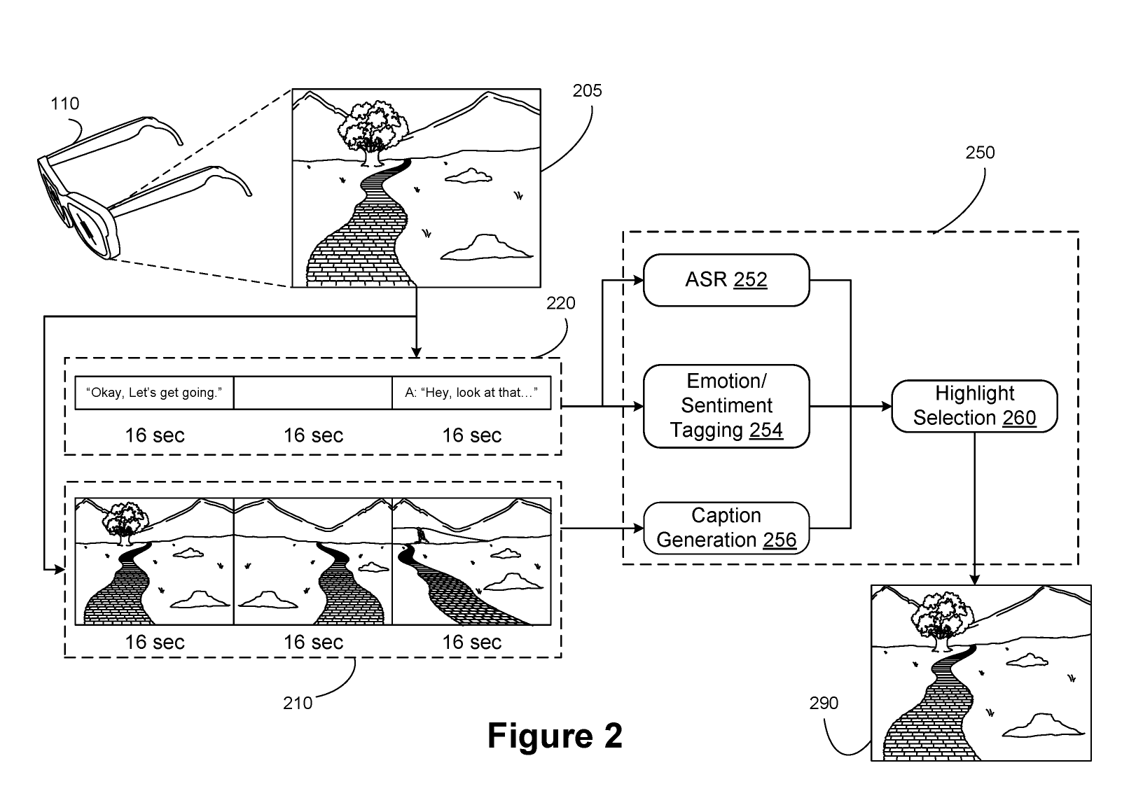

FIG. 2 illustrates an example diagram of the video highlight auto-capture feature automatically generating a highlight video 290 from video data 205 captured at the head-wearable device, in accordance with some embodiments. The video data 205 (e.g., the image data and the video data) is captured at the one or more imaging devices and the one or more audio devices of the head-wearable device 110. The video data is divided into video segments, comprising corresponding image segments 210 and audio segments 220 (e.g., each sixteen seconds in length), by one or more processors of the head-wearable device 110, the other device communicatively coupled to the head-wearable device 110, and/or the server device. The corresponding image segments 210 and audio segments 220 are sent to the multi-modal AI 250, executed at one or more processors of the head-wearable device 110, the other device, and/or the server device (e.g., the video data 205 may be captured at the head-wearable device 110, divided into segments at the other device, and processed by the multi-modal AI at the server device). In some embodiments, the multi-modal AI 250 includes ASR 252 which transcribes each audio segment of the audio segments 220 into respective transcriptions, determines a respective amount of background noise in each audio segment of the audio segments 220 (e.g., as a percentage of a total audio in each audio segment), identifies a respective number of speakers in each audio segment of the audio segments 220, and/or identifies a respective number of spoken words in each audio segment of the audio segments 220. In some embodiments, the multi-modal AI 250 performs emotion/sentiment tagging 254 to create a respective emotion/sentiment tag for each audio segment of the audio segments 220. In some embodiments, the multi-modal AI 250 includes computer vision and caption generation 256 which generates a respective caption describing each image segment of the image segments 210 (e.g., including one or more objects of interest). The multi-modal AI 250 determines, via a large language model (LLM) 260, whether each video segment (each pair of corresponding image segments 210 and audio segments 220) is a highlight segment (e.g., as described in reference to FIGS. 1A-1B) based on one or more of: (i) a respective transcription, (ii) a respective amount of background noise, (iii) a respective number of speakers, (iv) a respective number of spoken words, (v) a respective emotion/sentiment tag, and (vi) a respective caption describing a corresponding image segment. The multi-modal AI 250 then compiles all the highlight segments into a highlight video 290 (e.g., the highlight segments are compiled into a single video in a chronological order). In some embodiments, the highlight video 290 is presented to the user 101 (e.g., at the one or more displays and/or the one or more speakers of the head-wearable device 110 and/or the other device), stored at a computer memory device associated with the user 101 (e.g., a computer memory device of the head-wearable device 110 and/or the other device and/or a cloud storage device associated with the user 101), and/or sent to a device associated with another user (e.g., shared over social media, a messaging application, e-mail, etc.).

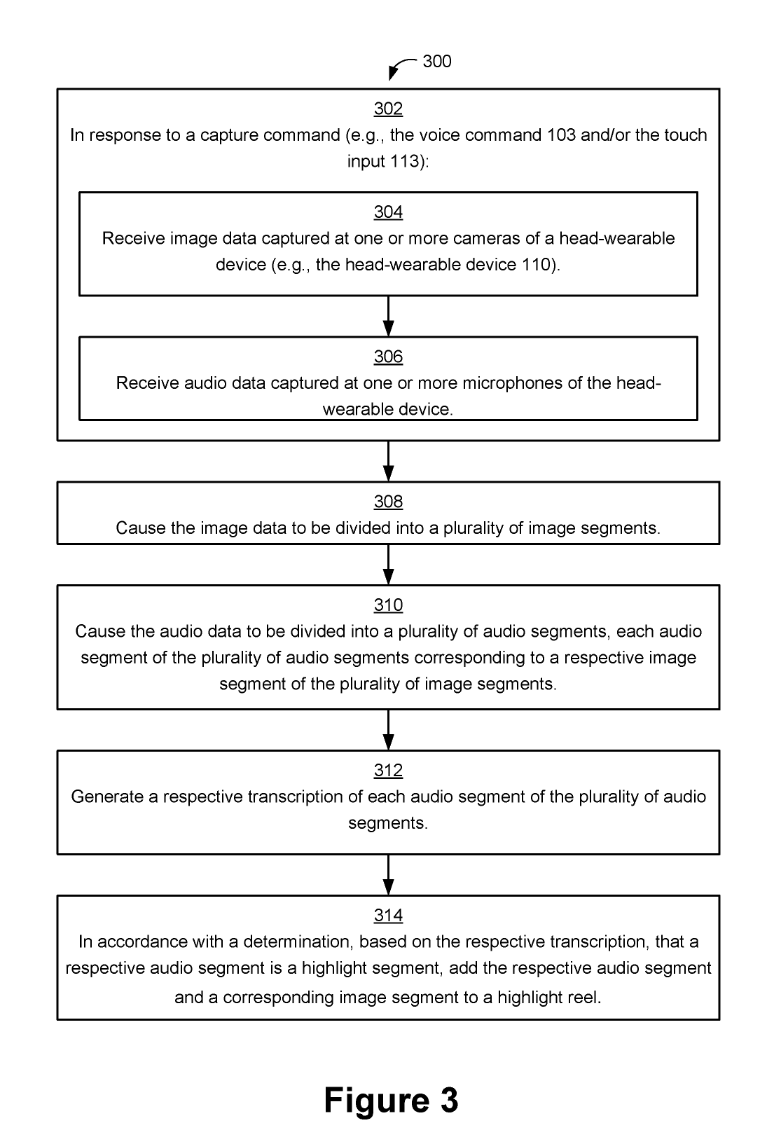

FIG. 3 illustrates a flow diagram of a method of automatically creating a highlight video from video data captured at a head-wearable device, in accordance with some embodiments. Operations (e.g., steps) of the method 300 can be performed by one or more processors (e.g., central processing unit and/or MCU) of a system. At least some of the operations shown in FIG. 3 correspond to instructions stored in a computer memory or computer-readable storage medium (e.g., storage, RAM, and/or memory) communicatively coupled to the head-wearable device 110. Operations of the method 300 can be performed by the head-wearable device 110 alone or in conjunction with one or more processors and/or hardware components of another communicatively coupled device and/or instructions stored in memory or computer-readable medium of the other device communicatively coupled to the head-wearable device 110. In some embodiments, the various operations of the methods described herein are interchangeable and/or optional, and respective operations of the methods are performed by any of the aforementioned devices, systems, or combination of devices and/or systems. For convenience, the method operations will be described below as being performed by particular component or device but should not be construed as limiting the performance of the operation to the particular device in all embodiments.

A1. FIG. 3 shows a flow chart of a method 300 for automatically creating a highlight video from video data captured at a head-wearable device, in accordance with some embodiments.

The method 300 occurs at head-wearable device (e.g., the head-wearable device 110) and/or another device communicatively coupled to the head-wearable device (e.g., the other device, as described in reference to FIGS. 1A-2) with one or more imaging devices (e.g., forward-facing cameras), one or more audio devices (e.g., microphones), one or more processors, and one or more computer-readable storage media including executable instructions that, when executed by the one or more processors, cause the one or more processors to perform operations corresponding to the method 300. In accordance with some embodiments, the method 300 includes, in response to a capture command (e.g., the capture command, as described in reference to FIGS. 1A-2, such as the voice command 103 and/or the touch input 113) (302): (i) receiving image data (e.g., the image data, as described in reference to FIGS. 1A-2) captured at one or more cameras of the head-wearable device and (ii) receiving audio data (e.g., the audio data, as described in reference to FIGS. 1A-2) captured at one or more microphones of the head-wearable device (e.g., the image data and the audio data comprise video data (e.g., the video segments, as described in reference to FIG. 1B, and/or the video data 205) captured at the head-wearable device). The method 300 further comprises: (i) causing the image data to be divided into a plurality of image segments (e.g., the eight image segments 122-192 and/or the image segments 210), (ii) causing the audio data to be divided into a plurality of audio segments (e.g., the eight audio segments 124-194 and/or the audio segments 220), each audio segment of the plurality of audio segments corresponding to a respective image segment of the plurality of image segments, and (iii) generating a respective transcription (e.g., the respective transcriptions, as described in reference to FIGS. 1A-2) of each audio segment of the plurality of audio segments. The method 300 further includes, in accordance with a determination, based on the respective transcription, that a respective audio segment is a highlight segment (e.g., the highlight segments, as described in reference to FIGS. 1A-2), add the respective audio segment and a corresponding image segment (e.g., the respective audio segment and the corresponding image segment comprise a video segment (e.g., the video segments, as described in reference to FIGS. 1A-2)) to a highlight video (e.g., the highlight video 290).

A2. In some embodiments of A2, the method 300 further includes, before adding the respective audio segment and the corresponding image segment to the highlight video, determining a respective sentiment tag (e.g., the one or more emotion/sentiment tags, as described in reference to FIGS. 1A-1B, and/or the emotion/sentiment tagging 254) of each audio segment of the plurality of audio segments. Additionally, the determination that the respective audio segment is a highlight segment is further based on the respective sentiment tag.

A3. In some embodiments of any of A1-A2, the method 300 further includes, before adding the respective audio segment and the corresponding image segment to the highlight video, identifying one or more respective objects (e.g., the one or more objects of interest, as described in reference to FIGS. 1A-1B, and/or the computer vision and caption generation 256) in each image segment of the plurality of image segments. Additionally, the determination that the respective audio segment is a highlight segment is further based on the one or more respective objects in the corresponding image segment.

A4. In some embodiments of any of A1-A3, the one or more respective objects are based on the capture command (e.g., as described in reference to FIGS. 1A-1B).

A5. In some embodiments of any of A1-A4, identifying one or more respective objects in each image segment of the plurality of image segments includes: (i) generating a respective caption for each image segment (e.g., via the computer vision and caption generation 256) of the plurality of image segments and (ii) determining that each respective caption describes the one or more respective objects. Additionally, the determination that the respective audio segment is a highlight segment is further based on the respective caption of the corresponding image segment.

A6. In some embodiments of any of A1-A5, the respective transcription of each audio segment of the plurality of audio segments is generated by an automatic speech recognition machine-learning model (e.g., ASR 252), based on the respective audio segment of the plurality of audio segments.

A7. In some embodiments of any of A1-A6, the determination that the respective audio segment is a highlight segment includes a determination (e.g., by the multi-modal AI 250) that the respective transcription satisfies a word number threshold (e.g., the word threshold, as described in reference to FIGS. 1A-1B).

A8. In some embodiments of any of A1-A7, the determination that the respective audio segment is a highlight segment includes a determination (e.g., by the multi-modal AI 250) that the respective audio segment satisfies a background noise threshold (e.g., the background noise threshold, as described in reference to FIGS. 1A-1B).

A9. In some embodiments of any of A1-A8, generating the respective transcription of each audio segment of the plurality of audio segments includes identifying (e.g., by the ASR 252) one or more speakers in each audio segment of the plurality of audio segments. Additionally, the determination that the respective audio segment is a highlight segment includes a determination (e.g., by the multi-modal AI 250) that the respective audio segment includes a threshold number of speakers (e.g., the speaker threshold, as described in reference to FIGS. 1A-1B).

A10. In some embodiments of any of A1-A9, the determination that the respective audio segment is a highlight segment includes a determination (e.g., by the ASR 252) that the respective audio segment includes one or more keywords (e.g., the one or more key words and/or key phrases, as described in reference to FIGS. 1A-1B).

A11. In some embodiments of any of A1-A10, the one or more keywords are based on the capture command (e.g., as described in reference to FIGS. 1A-1B).

A12. In some embodiments of any of A1-A11, the method 300 further includes, (i) in response to the capture command, receiving health signal data (e.g., heart rate, pupil dilation, breathing rate, etc.) captured at one or more health sensors (e.g., a body microphone, an eye-tracking device, an IMU sensor, etc.) of the head-wearable device and (ii) causing the health signal data to be divided into a plurality of health signal segments, each health signal segment of the plurality of health signal segments corresponding to a respective image segment of the plurality of image segments and a respective audio segment of the plurality of audio segments. The determination that the respective audio segment is a highlight segment is further based on the respective health signal segment (e.g., the respective health signal segment indicates that the user 101 is excited and/or relaxed).

A13. In some embodiments of any of A1-A12, the method 300 further includes, (i) in response to the capture command, receiving gaze data captured at one or more gaze-tracking devices (e.g., an eye-tracking camera and/or an IMU sensor providing IMU data that is used to approximate gaze location) of the head-wearable device and (ii) causing the gaze data to be divided into a plurality of gaze segments, each gaze segment of the plurality of gaze segments corresponding to a respective image segment of the plurality of image segments and a respective audio segment of the plurality of audio segments. The determination that the respective audio segment is a highlight segment is further based on the respective gaze segment (e.g., the respective gaze segment indicates that the user 101 is excited and/or focusing on an object).

A14. In some embodiments of any of A1-A13, the method 300 further includes, in accordance with a determination, based on the respective transcription, that another respective audio segment of the plurality of audio segments is not a highlight segment, discarding the other respective audio segment and another corresponding image segment of the plurality of image segments.

A15. In some embodiments of any of A1-A14, the method 300 further includes, after adding the respective audio segment and the corresponding image segment to the highlight video, presenting the highlight video (e.g., the highlight video 290) to a user (e.g., the user 101) (e.g., at the one or more displays and/or the one or more speakers of the head-wearable device 110, the other device, and/or the additional device communicatively coupled to the head-wearable device 110 and/or the other device).

A16. In some embodiments of any of A1-A15, the method 300 further includes, in response to an additional capture command (e.g., the capture command, as described in reference to FIGS. 1A-2, such as the voice command 103 and/or the touch input 113): (i) receiving additional image data (e.g., the image data, as described in reference to FIGS. 1A-2) captured at the one or more cameras of the head-wearable device and (ii) receiving additional audio data (e.g., the audio data, as described in reference to FIGS. 1A-2) captured at the one or more microphones of the head-wearable device. The method 300 further comprises: (i) causing the additional image data to be divided into a plurality of additional image segments (e.g., the eight image segments 122-192 and/or the image segments 210), (ii) causing the additional audio data to be divided into a plurality of additional audio segments (e.g., the eight audio segments 124-194 and/or the audio segments 220), each audio additional segment of the plurality of additional audio segments corresponding to a respective additional image segment of the plurality of additional image segments, and (iii) generating a respective additional transcription (e.g., the respective transcriptions, as described in reference to FIGS. 1A-2) of each additional audio segment of the plurality of additional audio segments. The method 300 further includes, in accordance with a determination, based on the respective transcription, that a respective audio segment is a highlight segment (e.g., the highlight segments, as described in reference to FIGS. 1A-2), add the respective audio segment and a corresponding image segment (e.g., the respective audio segment and the corresponding image segment comprise a video segment (e.g., the video segments, as described in reference to FIGS. 1A-2)) to a highlight video (e.g., the highlight video 290).

A17. In some embodiments of any of A1-A16, the head-wearable device is a pair of smart glasses.

A18. In some embodiments of any of A1-A17, generating the respective transcription of each audio segment of the plurality of audio segments is performed by a multi-modal AI (e.g., the multi-modal AI 250). Additionally, the determination, based on the respective transcription, that the respective audio segment is a highlight segment is made by the multi-modal AI.

B1. In accordance with some embodiments, a non-transitory computer readable storage medium including instructions that, when executed by one or more processors in communication with a head-wearable device (e.g., the head-wearable device 110), cause the one or more processors to perform operations corresponding to any of A1-A18.

C1. In accordance with some embodiments, a system that includes a head-wearable device (e.g., the head-wearable device 110), and the system is configured to perform operations corresponding to any of A1-A18.

D1. In accordance with some embodiments, means for performing or causing performance of the operations corresponding to any of A1-A18.

E1. A head-wearable device (e.g., the head-wearable device 110) configured to perform or cause performance of the operations corresponding to any of A1-A18.

F1. An intermediary processing device configured to offload processing operations for a head-wearable device (e.g., the head-wearable device 110) and configured to perform or cause performance of the operations corresponding to any of A1-A18.

### Example Extended-Reality Systems

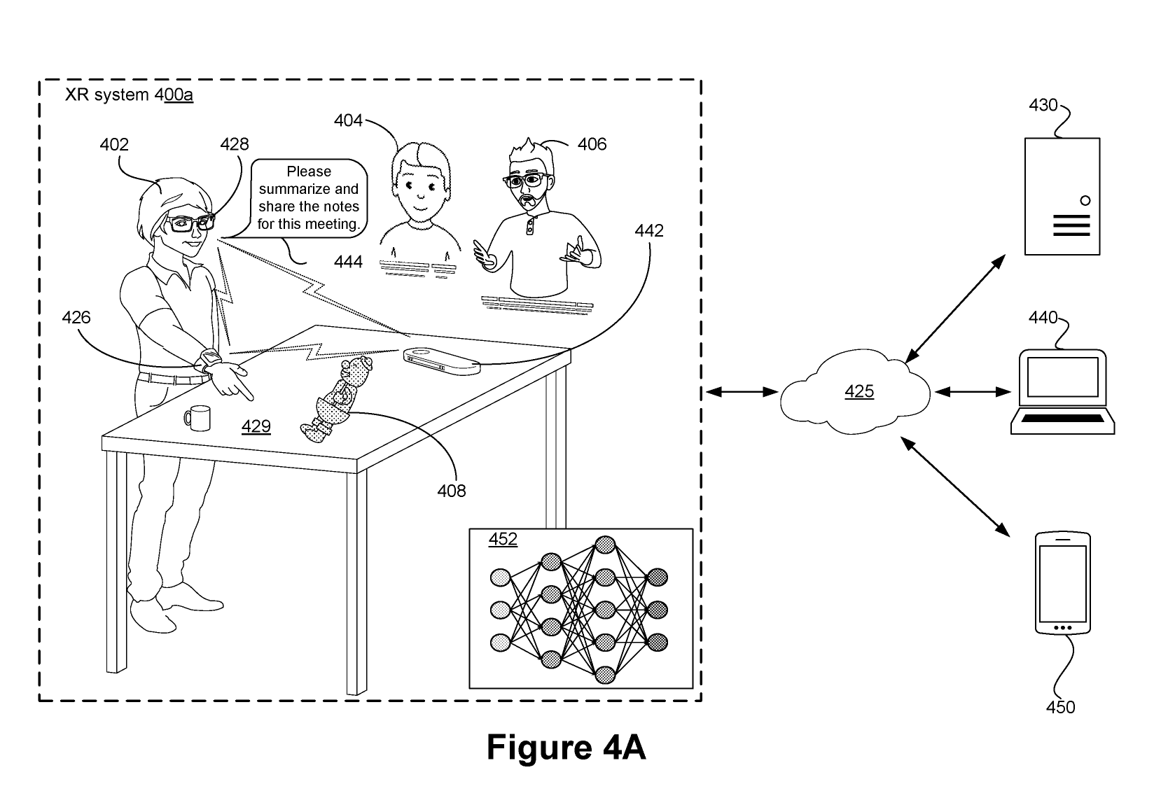

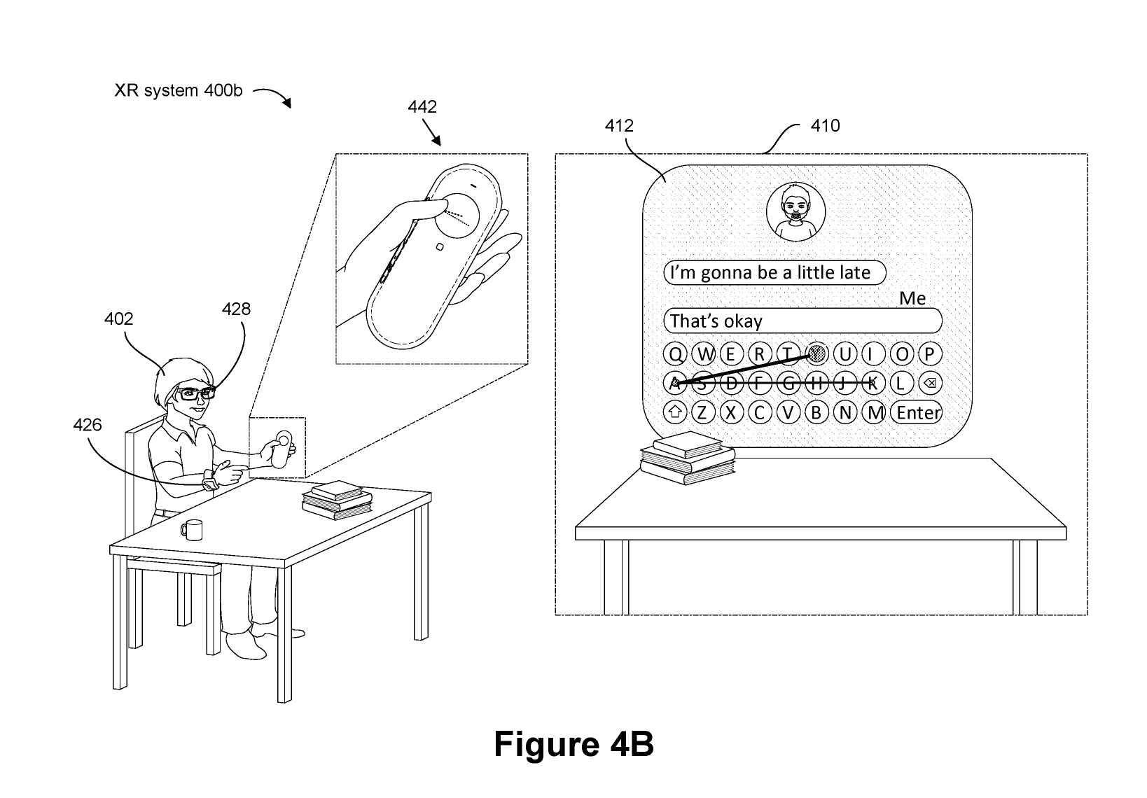

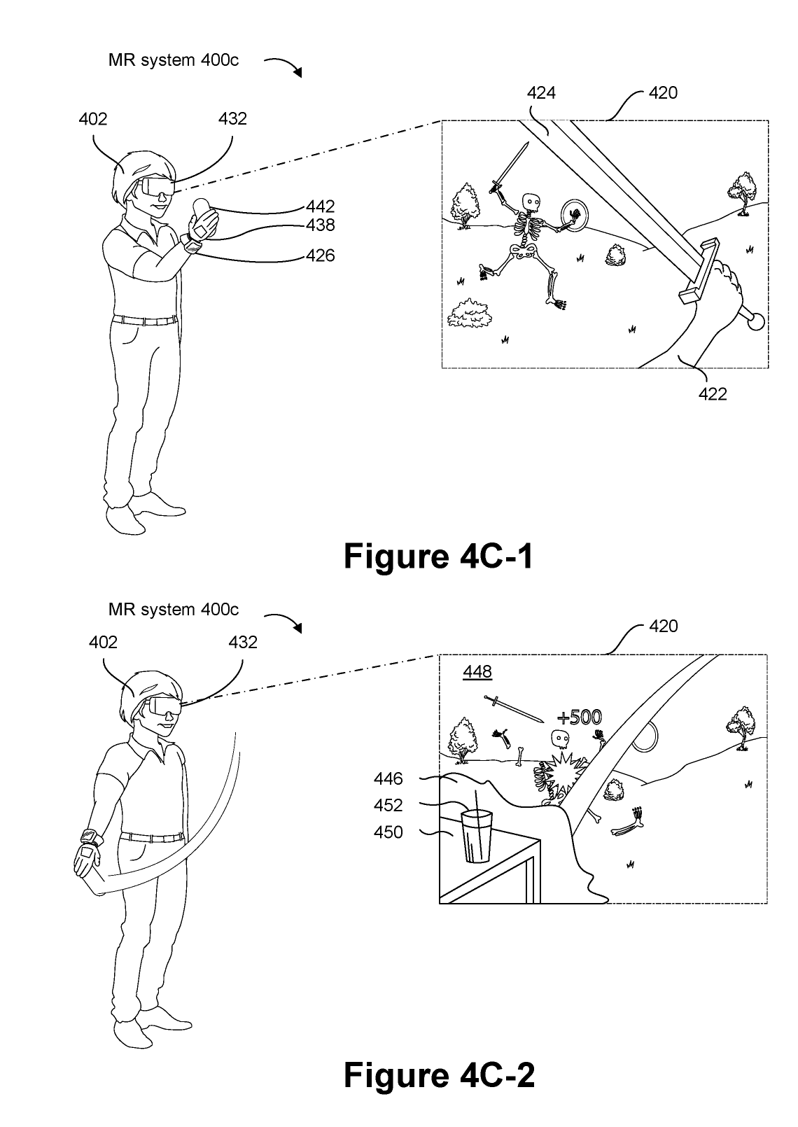

FIGS. 4A, 4B, 4C-1 and 4C-2, illustrate example XR systems that include AR and MR systems, in accordance with some embodiments. FIG. 4A shows a first XR system 400a and first example user interactions using a wrist-wearable device 426, a head-wearable device (e.g., AR device 428), and/or a HIPD 442. FIG. 4B shows a second XR system 400b and second example user interactions using a wrist-wearable device 426, AR device 428, and/or an HIPD 442. FIGS. 4C-1 and 4C-2 show a third MR system 400c and third example user interactions using a wrist-wearable device 426, a head-wearable device (e.g., an MR device such as a VR device), and/or an HIPD 442. As the skilled artisan will appreciate upon reading the descriptions provided herein, the above-example AR and MR systems (described in detail below) can perform various functions and/or operations.

The wrist-wearable device 426, the head-wearable devices, and/or the HIPD 442 can communicatively couple via a network 425 (e.g., cellular, near field, Wi-Fi, personal area network, wireless LAN). Additionally, the wrist-wearable device 426, the head-wearable device, and/or the HIPD 442 can also communicatively couple with one or more servers 430, computers 440 (e.g., laptops, computers), mobile devices 450 (e.g., smartphones, tablets), and/or other electronic devices via the network 425 (e.g., cellular, near field, Wi-Fi, personal area network, wireless LAN). Similarly, a smart textile-based garment, when used, can also communicatively couple with the wrist-wearable device 426, the head-wearable device(s), the HIPD 442, the one or more servers 430, the computers 440, the mobile devices 450, and/or other electronic devices via the network 425 to provide inputs.

Turning to FIG. 4A, a user 402 is shown wearing the wrist-wearable device 426 and the AR device 428 and having the HIPD 442 on their desk. The wrist-wearable device 426, the AR device 428, and the HIPD 442 facilitate user interaction with an AR environment. In particular, as shown by the first AR system 400a, the wrist-wearable device 426, the AR device 428, and/or the HIPD 442 cause presentation of one or more avatars 404, digital representations of contacts 406, and virtual objects 408. As discussed below, the user 402 can interact with the one or more avatars 404, digital representations of the contacts 406, and virtual objects 408 via the wrist-wearable device 426, the AR device 428, and/or the HIPD 442. In addition, the user 402 is also able to directly view physical objects in the environment, such as a physical table 429, through transparent lens(es) and waveguide(s) of the AR device 428. Alternatively, an MR device could be used in place of the AR device 428 and a similar user experience can take place, but the user would not be directly viewing physical objects in the environment, such as table 429, and would instead be presented with a virtual reconstruction of the table 429 produced from one or more sensors of the MR device (e.g., an outward facing camera capable of recording the surrounding environment).

The user 402 can use any of the wrist-wearable device 426, the AR device 428 (e.g., through physical inputs at the AR device and/or built-in motion tracking of a user’s extremities), a smart-textile garment, externally mounted extremity tracking device, the HIPD 442 to provide user inputs, etc. For example, the user 402 can perform one or more hand gestures that are detected by the wrist-wearable device 426 (e.g., using one or more EMG sensors and/or IMUs built into the wrist-wearable device) and/or AR device 428 (e.g., using one or more image sensors or cameras) to provide a user input. Alternatively, or additionally, the user 402 can provide a user input via one or more touch surfaces of the wrist-wearable device 426, the AR device 428, and/or the HIPD 442, and/or voice commands captured by a microphone of the wrist-wearable device 426, the AR device 428, and/or the HIPD 442. The wrist-wearable device 426, the AR device 428, and/or the HIPD 442 include an artificially intelligent digital assistant to help the user in providing a user input (e.g., completing a sequence of operations, suggesting different operations or commands, providing reminders, confirming a command). For example, the digital assistant can be invoked through an input occurring at the AR device 428 (e.g., via an input at a temple arm of the AR device 428). In some embodiments, the user 402 can provide a user input via one or more facial gestures and/or facial expressions. For example, cameras of the wrist-wearable device 426, the AR device 428, and/or the HIPD 442 can track the user 402’s eyes for navigating a user interface.

The wrist-wearable device 426, the AR device 428, and/or the HIPD 442 can operate alone or in conjunction to allow the user 402 to interact with the AR environment. In some embodiments, the HIPD 442 is configured to operate as a central hub or control center for the wrist-wearable device 426, the AR device 428, and/or another communicatively coupled device. For example, the user 402 can provide an input to interact with the AR environment at any of the wrist-wearable device 426, the AR device 428, and/or the HIPD 442, and the HIPD 442 can identify one or more back-end and front-end tasks to cause the performance of the requested interaction and distribute instructions to cause the performance of the one or more back-end and front-end tasks at the wrist-wearable device 426, the AR device 428, and/or the HIPD 442. In some embodiments, a back-end task is a background-processing task that is not perceptible by the user (e.g., rendering content, decompression, compression, application-specific operations), and a front-end task is a user-facing task that is perceptible to the user (e.g., presenting information to the user, providing feedback to the user). The HIPD 442 can perform the back-end tasks and provide the wrist-wearable device 426 and/or the AR device 428 operational data corresponding to the performed back-end tasks such that the wrist-wearable device 426 and/or the AR device 428 can perform the front-end tasks. In this way, the HIPD 442, which has more computational resources and greater thermal headroom than the wrist-wearable device 426 and/or the AR device 428, performs computationally intensive tasks and reduces the computer resource utilization and/or power usage of the wrist-wearable device 426 and/or the AR device 428.

In the example shown by the first AR system 400a, the HIPD 442 identifies one or more back-end tasks and front-end tasks associated with a user request to initiate an AR video call with one or more other users (represented by the avatar 404 and the digital representation of the contact 406) and distributes instructions to cause the performance of the one or more back-end tasks and front-end tasks. In particular, the HIPD 442 performs back-end tasks for processing and/or rendering image data (and other data) associated with the AR video call and provides operational data associated with the performed back-end tasks to the AR device 428 such that the AR device 428 performs front-end tasks for presenting the AR video call (e.g., presenting the avatar 404 and the digital representation of the contact 406).

In some embodiments, the HIPD 442 can operate as a focal or anchor point for causing the presentation of information. This allows the user 402 to be generally aware of where information is presented. For example, as shown in the first AR system 400a, the avatar 404 and the digital representation of the contact 406 are presented above the HIPD 442. In particular, the HIPD 442 and the AR device 428 operate in conjunction to determine a location for presenting the avatar 404 and the digital representation of the contact 406. In some embodiments, information can be presented within a predetermined distance from the HIPD 442 (e.g., within five meters). For example, as shown in the first AR system 400a, virtual object 408 is presented on the desk some distance from the HIPD 442. Similar to the above example, the HIPD 442 and the AR device 428 can operate in conjunction to determine a location for presenting the virtual object 408. Alternatively, in some embodiments, presentation of information is not bound by the HIPD 442. More specifically, the avatar 404, the digital representation of the contact 406, and the virtual object 408 do not have to be presented within a predetermined distance of the HIPD 442. While an AR device 428 is described working with an HIPD, an MR headset can be interacted with in the same way as the AR device 428.

User inputs provided at the wrist-wearable device 426, the AR device 428, and/or the HIPD 442 are coordinated such that the user can use any device to initiate, continue, and/or complete an operation. For example, the user 402 can provide a user input to the AR device 428 to cause the AR device 428 to present the virtual object 408 and, while the virtual object 408 is presented by the AR device 428, the user 402 can provide one or more hand gestures via the wrist-wearable device 426 to interact and/or manipulate the virtual object 408. While an AR device 428 is described working with a wrist-wearable device 426, an MR headset can be interacted with in the same way as the AR device 428.

### Integration of Artificial Intelligence with XR Systems

FIG. 4A illustrates an interaction in which an artificially intelligent virtual assistant can assist in requests made by a user 402. The AI virtual assistant can be used to complete open-ended requests made through natural language inputs by a user 402. For example, in FIG. 4A the user 402 makes an audible request 444 to summarize the conversation and then share the summarized conversation with others in the meeting. In addition, the AI virtual assistant is configured to use sensors of the XR system (e.g., cameras of an XR headset, microphones, and various other sensors of any of the devices in the system) to provide contextual prompts to the user for initiating tasks.

FIG. 4A also illustrates an example neural network 452 used in Artificial Intelligence applications. Uses of Artificial Intelligence (AI) are varied and encompass many different aspects of the devices and systems described herein. AI capabilities cover a diverse range of applications and deepen interactions between the user 402 and user devices (e.g., the AR device 428, an MR device 432, the HIPD 442, the wrist-wearable device 426). The AI discussed herein can be derived using many different training techniques. While the primary AI model example discussed herein is a neural network, other AI models can be used. Non-limiting examples of AI models include artificial neural networks (ANNs), deep neural networks (DNNs), convolution neural networks (CNNs), recurrent neural networks (RNNs), large language models (LLMs), long short-term memory networks, transformer models, decision trees, random forests, support vector machines, k-nearest neighbors, genetic algorithms, Markov models, Bayesian networks, fuzzy logic systems, and deep reinforcement learnings, etc. The AI models can be implemented at one or more of the user devices, and/or any other devices described herein. For devices and systems herein that employ multiple AI models, different models can be used depending on the task. For example, for a natural-language artificially intelligent virtual assistant, an LLM can be used and for the object detection of a physical environment, a DNN can be used instead.

In another example, an AI virtual assistant can include many different AI models and based on the user’s request, multiple AI models may be employed (concurrently, sequentially or a combination thereof). For example, an LLM-based AI model can provide instructions for helping a user follow a recipe and the instructions can be based in part on another AI model that is derived from an ANN, a DNN, an RNN, etc. that is capable of discerning what part of the recipe the user is on (e.g., object and scene detection).

As AI training models evolve, the operations and experiences described herein could potentially be performed with different models other than those listed above, and a person skilled in the art would understand that the list above is non-limiting.

A user 402 can interact with an AI model through natural language inputs captured by a voice sensor, text inputs, or any other input modality that accepts natural language and/or a corresponding voice sensor module. In another instance, input is provided by tracking the eye gaze of a user 402 via a gaze tracker module. Additionally, the AI model can also receive inputs beyond those supplied by a user 402. For example, the AI can generate its response further based on environmental inputs (e.g., temperature data, image data, video data, ambient light data, audio data, GPS location data, inertial measurement (i.e., user motion) data, pattern recognition data, magnetometer data, depth data, pressure data, force data, neuromuscular data, heart rate data, temperature data, sleep data) captured in response to a user request by various types of sensors and/or their corresponding sensor modules. The sensors’ data can be retrieved entirely from a single device (e.g., AR device 428) or from multiple devices that are in communication with each other (e.g., a system that includes at least two of an AR device 428, an MR device 432, the HIPD 442, the wrist-wearable device 426, etc.). The AI model can also access additional information (e.g., one or more servers 430, the computers 440, the mobile devices 450, and/or other electronic devices) via a network 425.

A non-limiting list of AI-enhanced functions includes but is not limited to image recognition, speech recognition (e.g., automatic speech recognition), text recognition (e.g., scene text recognition), pattern recognition, natural language processing and understanding, classification, regression, clustering, anomaly detection, sequence generation, content generation, and optimization. In some embodiments, AI-enhanced functions are fully or partially executed on cloud-computing platforms communicatively coupled to the user devices (e.g., the AR device 428, an MR device 432, the HIPD 442, the wrist-wearable device 426) via the one or more networks. The cloud-computing platforms provide scalable computing resources, distributed computing, managed AI services, interference acceleration, pre-trained models, APIs and/or other resources to support comprehensive computations required by the AI-enhanced function.

Example outputs stemming from the use of an AI model can include natural language responses, mathematical calculations, charts displaying information, audio, images, videos, texts, summaries of meetings, predictive operations based on environmental factors, classifications, pattern recognitions, recommendations, assessments, or other operations. In some embodiments, the generated outputs are stored on local memories of the user devices (e.g., the AR device 428, an MR device 432, the HIPD 442, the wrist-wearable device 426), storage options of the external devices (servers, computers, mobile devices, etc.), and/or storage options of the cloud-computing platforms.

The AI-based outputs can be presented across different modalities (e.g., audio-based, visual-based, haptic-based, and any combination thereof) and across different devices of the XR system described herein. Some visual-based outputs can include the displaying of information on XR augments of an XR headset, user interfaces displayed at a wrist-wearable device, laptop device, mobile device, etc. On devices with or without displays (e.g., HIPD 442), haptic feedback can provide information to the user 402. An AI model can also use the inputs described above to determine the appropriate modality and device(s) to present content to the user (e.g., a user walking on a busy road can be presented with an audio output instead of a visual output to avoid distracting the user 402).

### Example Augmented Reality Interaction

FIG. 4B shows the user 402 wearing the wrist-wearable device 426 and the AR device 428 and holding the HIPD 442. In the second AR system 400b, the wrist-wearable device 426, the AR device 428, and/or the HIPD 442 are used to receive and/or provide one or more messages to a contact of the user 402. In particular, the wrist-wearable device 426, the AR device 428, and/or the HIPD 442 detect and coordinate one or more user inputs to initiate a messaging application and prepare a response to a received message via the messaging application.

In some embodiments, the user 402 initiates, via a user input, an application on the wrist-wearable device 426, the AR device 428, and/or the HIPD 442 that causes the application to initiate on at least one device. For example, in the second AR system 400b the user 402 performs a hand gesture associated with a command for initiating a messaging application (represented by messaging user interface 412); the wrist-wearable device 426 detects the hand gesture; and, based on a determination that the user 402 is wearing the AR device 428, causes the AR device 428 to present a messaging user interface 412 of the messaging application. The AR device 428 can present the messaging user interface 412 to the user 402 via its display (e.g., as shown by user 402’s field of view 410). In some embodiments, the application is initiated and can be run on the device (e.g., the wrist-wearable device 426, the AR device 428, and/or the HIPD 442) that detects the user input to initiate the application, and the device provides another device operational data to cause the presentation of the messaging application. For example, the wrist-wearable device 426 can detect the user input to initiate a messaging application, initiate and run the messaging application, and provide operational data to the AR device 428 and/or the HIPD 442 to cause presentation of the messaging application. Alternatively, the application can be initiated and run at a device other than the device that detected the user input. For example, the wrist-wearable device 426 can detect the hand gesture associated with initiating the messaging application and cause the HIPD 442 to run the messaging application and coordinate the presentation of the messaging application.

Further, the user 402 can provide a user input provided at the wrist-wearable device 426, the AR device 428, and/or the HIPD 442 to continue and/or complete an operation initiated at another device. For example, after initiating the messaging application via the wrist-wearable device 426 and while the AR device 428 presents the messaging user interface 412, the user 402 can provide an input at the HIPD 442 to prepare a response (e.g., shown by the swipe gesture performed on the HIPD 442). The user 402’s gestures performed on the HIPD 442 can be provided and/or displayed on another device. For example, the user 402’s swipe gestures performed on the HIPD 442 are displayed on a virtual keyboard of the messaging user interface 412 displayed by the AR device 428.

In some embodiments, the wrist-wearable device 426, the AR device 428, the HIPD 442, and/or other communicatively coupled devices can present one or more notifications to the user 402. The notification can be an indication of a new message, an incoming call, an application update, a status update, etc. The user 402 can select the notification via the wrist-wearable device 426, the AR device 428, or the HIPD 442 and cause presentation of an application or operation associated with the notification on at least one device. For example, the user 402 can receive a notification that a message was received at the wrist-wearable device 426, the AR device 428, the HIPD 442, and/or other communicatively coupled device and provide a user input at the wrist-wearable device 426, the AR device 428, and/or the HIPD 442 to review the notification, and the device detecting the user input can cause an application associated with the notification to be initiated and/or presented at the wrist-wearable device 426, the AR device 428, and/or the HIPD 442.

While the above example describes coordinated inputs used to interact with a messaging application, the skilled artisan will appreciate upon reading the descriptions that user inputs can be coordinated to interact with any number of applications including, but not limited to, gaming applications, social media applications, camera applications, web-based applications, financial applications, etc. For example, the AR device 428 can present to the user 402 game application data and the HIPD 442 can use a controller to provide inputs to the game. Similarly, the user 402 can use the wrist-wearable device 426 to initiate a camera of the AR device 428, and the user can use the wrist-wearable device 426, the AR device 428, and/or the HIPD 442 to manipulate the image capture (e.g., zoom in or out, apply filters) and capture image data.

While an AR device 428 is shown being capable of certain functions, it is understood that an AR device can be an AR device with varying functionalities based on costs and market demands. For example, an AR device may include a single output modality such as an audio output modality. In another example, the AR device may include a low-fidelity display as one of the output modalities, where simple information (e.g., text and/or low-fidelity images/video) is capable of being presented to the user. In yet another example, the AR device can be configured with face-facing light emitting diodes (LEDs) configured to provide a user with information, e.g., an LED around the right-side lens can illuminate to notify the wearer to turn right while directions are being provided or an LED on the left-side can illuminate to notify the wearer to turn left while directions are being provided. In another embodiment, the AR device can include an outward-facing projector such that information (e.g., text information, media) may be displayed on the palm of a user’s hand or other suitable surface (e.g., a table, whiteboard). In yet another embodiment, information may also be provided by locally dimming portions of a lens to emphasize portions of the environment in which the user’s attention should be directed. Some AR devices can present AR augments either monocularly or binocularly (e.g., an AR augment can be presented at only a single display associated with a single lens as opposed presenting an AR augmented at both lenses to produce a binocular image). In some instances an AR device capable of presenting AR augments binocularly can optionally display AR augments monocularly as well (e.g., for power-saving purposes or other presentation considerations). These examples are non-exhaustive and features of one AR device described above can be combined with features of another AR device described above. While features and experiences of an AR device have been described generally in the preceding sections, it is understood that the described functionalities and experiences can be applied in a similar manner to an MR headset, which is described below in the proceeding sections.

### Example Mixed Reality Interaction

Turning to FIGS. 4C-1 and 4C-2, the user 402 is shown wearing the wrist-wearable device 426 and an MR device 432 (e.g., a device capable of providing either an entirely VR experience or an MR experience that displays object(s) from a physical environment at a display of the device) and holding the HIPD 442. In the third AR system 400c, the wrist-wearable device 426, the MR device 432, and/or the HIPD 442 are used to interact within an MR environment, such as a VR game or other MR/VR application. While the MR device 432 presents a representation of a VR game (e.g., first MR game environment 420) to the user 402, the wrist-wearable device 426, the MR device 432, and/or the HIPD 442 detect and coordinate one or more user inputs to allow the user 402 to interact with the VR game.

In some embodiments, the user 402 can provide a user input via the wrist-wearable device 426, the MR device 432, and/or the HIPD 442 that causes an action in a corresponding MR environment. For example, the user 402 in the third MR system 400c (shown in FIG. 4C-1) raises the HIPD 442 to prepare for a swing in the first MR game environment 420. The MR device 432, responsive to the user 402 raising the HIPD 442, causes the MR representation of the user 422 to perform a similar action (e.g., raise a virtual object, such as a virtual sword 424). In some embodiments, each device uses respective sensor data and/or image data to detect the user input and provide an accurate representation of the user 402’s motion. For example, image sensors (e.g., SLAM cameras or other cameras) of the HIPD 442 can be used to detect a position of the HIPD 442 relative to the user 402’s body such that the virtual object can be positioned appropriately within the first MR game environment 420; sensor data from the wrist-wearable device 426 can be used to detect a velocity at which the user 402 raises the HIPD 442 such that the MR representation of the user 422 and the virtual sword 424 are synchronized with the user 402’s movements; and image sensors of the MR device 432 can be used to represent the user 402’s body, boundary conditions, or real-world objects within the first MR game environment 420.

In FIG. 4C-2, the user 402 performs a downward swing while holding the HIPD 442. The user 402’s downward swing is detected by the wrist-wearable device 426, the MR device 432, and/or the HIPD 442 and a corresponding action is performed in the first MR game environment 420. In some embodiments, the data captured by each device is used to improve the user’s experience within the MR environment. For example, sensor data of the wrist-wearable device 426 can be used to determine a speed and/or force at which the downward swing is performed and image sensors of the HIPD 442 and/or the MR device 432 can be used to determine a location of the swing and how it should be represented in the first MR game environment 420, which, in turn, can be used as inputs for the MR environment (e.g., game mechanics, which can use detected speed, force, locations, and/or aspects of the user 402’s actions to classify a user’s inputs (e.g., user performs a light strike, hard strike, critical strike, glancing strike, miss) or calculate an output (e.g., amount of damage)).

FIG. 4C-2 further illustrates that a portion of the physical environment is reconstructed and displayed at a display of the MR device 432 while the MR game environment 420 is being displayed. In this instance, a reconstruction of the physical environment 446 is displayed in place of a portion of the MR game environment 420 when object(s) in the physical environment are potentially in the path of the user (e.g., a collision with the user and an object in the physical environment are likely). Thus, this example MR game environment 420 includes (i) an immersive VR portion 448 (e.g., an environment that does not have a corollary counterpart in a nearby physical environment) and (ii) a reconstruction of the physical environment 446 (e.g., table 450 and cup 452). While the example shown here is an MR environment that shows a reconstruction of the physical environment to avoid collisions, other uses of reconstructions of the physical environment can be used, such as defining features of the virtual environment based on the surrounding physical environment (e.g., a virtual column can be placed based on an object in the surrounding physical environment (e.g., a tree)).

While the wrist-wearable device 426, the MR device 432, and/or the HIPD 442 are described as detecting user inputs, in some embodiments, user inputs are detected at a single device (with the single device being responsible for distributing signals to the other devices for performing the user input). For example, the HIPD 442 can operate an application for generating the first MR game environment 420 and provide the MR device 432 with corresponding data for causing the presentation of the first MR game environment 420, as well as detect the user 402’s movements (while holding the HIPD 442) to cause the performance of corresponding actions within the first MR game environment 420. Additionally or alternatively, in some embodiments, operational data (e.g., sensor data, image data, application data, device data, and/or other data) of one or more devices is provided to a single device (e.g., the HIPD 442) to process the operational data and cause respective devices to perform an action associated with processed operational data.

In some embodiments, the user 402 can wear a wrist-wearable device 426, wear an MR device 432, wear smart textile-based garments 438 (e.g., wearable haptic gloves), and/or hold an HIPD 442 device. In this embodiment, the wrist-wearable device 426, the MR device 432, and/or the smart textile-based garments 438 are used to interact within an MR environment (e.g., any AR or MR system described above in reference to FIGS. 4A–4B). While the MR device 432 presents a representation of an MR game (e.g., second MR game environment 420) to the user 402, the wrist-wearable device 426, the MR device 432, and/or the smart textile-based garments 438 detect and coordinate one or more user inputs to allow the user 402 to interact with the MR environment.

In some embodiments, the user 402 can provide a user input via the wrist-wearable device 426, an HIPD 442, the MR device 432, and/or the smart textile-based garments 438 that causes an action in a corresponding MR environment. In some embodiments, each device uses respective sensor data and/or image data to detect the user input and provide an accurate representation of the user 402’s motion. While four different input devices are shown (e.g., a wrist-wearable device 426, an MR device 432, an HIPD 442, and a smart textile-based garment 438) each one of these input devices entirely on its own can provide inputs for fully interacting with the MR environment. For example, the wrist-wearable device can provide sufficient inputs on its own for interacting with the MR environment. In some embodiments, if multiple input devices are used (e.g., a wrist-wearable device and the smart textile-based garment 438) sensor fusion can be utilized to ensure inputs are correct. While multiple input devices are described, it is understood that other input devices can be used in conjunction or on their own instead, such as but not limited to external motion-tracking cameras, other wearable devices fitted to different parts of a user, apparatuses that allow for a user to experience walking in an MR environment while remaining substantially stationary in the physical environment, etc.

As described above, the data captured by each device is used to improve the user’s experience within the MR environment. Although not shown, the smart textile-based garments 438 can be used in conjunction with an MR device and/or an HIPD 442.

While some experiences are described as occurring on an AR device and other experiences are described as occurring on an MR device, one skilled in the art would appreciate that experiences can be ported over from an MR device to an AR device, and vice versa.

Some definitions of devices and components that can be included in some or all of the example devices discussed are defined here for ease of reference. A skilled artisan will appreciate that certain types of the components described may be more suitable for a particular set of devices, and less suitable for a different set of devices. But subsequent reference to the components defined here should be considered to be encompassed by the definitions provided.

In some embodiments example devices and systems, including electronic devices and systems, will be discussed. Such example devices and systems are not intended to be limiting, and one of skill in the art will understand that alternative devices and systems to the example devices and systems described herein may be used to perform the operations and construct the systems and devices that are described herein.

As described herein, an electronic device is a device that uses electrical energy to perform a specific function. It can be any physical object that contains electronic components such as transistors, resistors, capacitors, diodes, and integrated circuits. Examples of electronic devices include smartphones, laptops, digital cameras, televisions, gaming consoles, and music players, as well as the example electronic devices discussed herein. As described herein, an intermediary electronic device is a device that sits between two other electronic devices, and/or a subset of components of one or more electronic devices and facilitates communication, and/or data processing and/or data transfer between the respective electronic devices and/or electronic components.

The foregoing descriptions of FIGS. 4A–4C-2 provided above are intended to augment the description provided in reference to FIGS. 1A-3. While terms in the following description may not be identical to terms used in the foregoing description, a person having ordinary skill in the art would understand these terms to have the same meaning.

Any data collection performed by the devices described herein and/or any devices configured to perform or cause the performance of the different embodiments described above in reference to any of the Figures, hereinafter the “devices,” is done with user consent and in a manner that is consistent with all applicable privacy laws. Users are given options to allow the devices to collect data, as well as the option to limit or deny collection of data by the devices. A user is able to opt in or opt out of any data collection at any time. Further, users are given the option to request the removal of any collected data.

It will be understood that, although the terms “first,” “second,” etc. may be used herein to describe various elements, these elements should not be limited by these terms. These terms are only used to distinguish one element from another.

The terminology used herein is for the purpose of describing particular embodiments only and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” can be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” can be construed to mean “upon determining” or “in response to determining” or “in accordance with a determination” or “upon detecting” or “in response to detecting” that the stated condition precedent is true, depending on the context.

The foregoing description, for purpose of explanation, has been described with reference to specific embodiments. However, the illustrative discussions above are not intended to be exhaustive or to limit the claims to the precise forms disclosed. Many modifications and variations are possible in view of the above teachings. The embodiments were chosen and described in order to best explain principles of operation and practical applications, to thereby enable others skilled in the art.

What is claimed is:

1. A non-transitory, computer-readable storage medium including executable instructions that, when executed by one or more processors, cause the one or more processors to:
in response to a capture command:
receive image data captured at one or more cameras of a head-wearable device; and
receive audio data captured at one or more microphones of the head-wearable device;
cause the image data to be divided into a plurality of image segments;
cause the audio data to be divided into a plurality of audio segments, each audio segment of the plurality of audio segments corresponding to a respective image segment of the plurality of image segments;
generate a respective transcription of each audio segment of the plurality of audio segments;
in accordance with a determination, based on the respective transcription, that a respective audio segment is a highlight segment, add the respective audio segment and a corresponding image segment to a highlight video.

2. The non-transitory, computer-readable storage medium of claim 1, wherein the executable instructions further cause the one or more processors to:
after adding the respective audio segment and the corresponding image segment to the highlight video, present the highlight video to a user.

3. The non-transitory, computer-readable storage medium of claim 1, wherein
the executable instructions further cause the one or more processors to, before adding the respective audio segment and the corresponding image segment to the highlight video, identify one or more respective objects in each image segment of the plurality of image segments; and
the determination that the respective audio segment is a highlight segment is further based on the one or more respective objects in the corresponding image segment.

4. The non-transitory, computer-readable storage medium of claim 3, wherein the one or more respective objects are based on the capture command.

5. The non-transitory, computer-readable storage medium of claim 3, wherein:
identifying the one or more respective objects in each image segment of the plurality of image segments includes:
generating a respective caption for each image segment of the plurality of image segments; and
determining that each respective caption describes the one or more respective objects; and
the determination that the respective audio segment is a highlight segment is further based on the respective caption of the corresponding image segment.

6. The non-transitory, computer-readable storage medium of claim 1, wherein:
the executable instructions further cause the one or more processors to, before adding the respective audio segment and the corresponding image segment to the highlight video, determine a respective sentiment tag of each audio segment of the plurality of audio segments; and
the determination that the respective audio segment is a highlight segment is further based on the respective sentiment tag.

7. The non-transitory, computer-readable storage medium of claim 1, wherein the determination that the respective audio segment is a highlight segment includes a determination that the respective transcription satisfies a word number threshold.

8. The non-transitory, computer-readable storage medium of claim 1, wherein the determination that the respective audio segment is a highlight segment includes a determination that the respective audio segment satisfies a background noise threshold.

9. The non-transitory, computer-readable storage medium of claim 1, wherein:
generating the respective transcription of each audio segment of the plurality of audio segments includes identifying one or more speakers in each audio segment of the plurality of audio segments; and
the determination that the respective audio segment is a highlight segment includes a determination that the respective audio segment includes a threshold number of speakers.

10. The non-transitory, computer-readable storage medium of claim 1, wherein the determination that the respective audio segment is a highlight segment includes a determination that the respective audio segment includes one or more keywords.

11. The non-transitory, computer-readable storage medium of claim 10, wherein the one or more keywords are based on the capture command.

12. The non-transitory, computer-readable storage medium of claim 1, wherein the executable instructions further cause the one or more processors to:
in response to the capture command, receive health signal data captured at one or more health sensors of the head-wearable device; and
cause the health signal data to be divided into a plurality of health signal segments, each respective health signal segment of the plurality of health signal segments corresponding to a respective image segment of the plurality of image segments and a respective audio segment of the plurality of audio segments, wherein:
the determination that the respective audio segment is a highlight segment is further based on the respective health signal segment.

13. The non-transitory, computer-readable storage medium of claim 1, wherein the executable instructions further cause the one or more processors to:
in response to the capture command, receive gaze data captured at one or more gaze-tracking devices of the head-wearable device; and
cause the gaze data to be divided into a plurality of gaze segments, each respective gaze segment of the plurality of gaze segments corresponding to a respective image segment of the plurality of image segments and a respective audio segment of the plurality of audio segments, wherein:
the determination that the respective audio segment is a highlight segment is further based on the respective gaze segment.

14. The non-transitory, computer-readable storage medium of claim 1, wherein the executable instructions further cause the one or more processors to:
in response to an additional capture command:
receive additional image data captured at the one or more cameras of the head-wearable device; and
receive additional audio data captured at the one or more microphones of the head-wearable device;
cause the additional image data to be divided into a plurality of additional image segments;
cause the additional audio data to be divided into a plurality of additional audio segments, each additional audio segment of the plurality of additional audio segments corresponding to a respective additional image segment of the plurality of additional image segments;
generate a respective additional transcription of each additional audio segment of the plurality of additional audio segments;
in accordance with a determination, based on the respective additional transcription, that a respective additional audio segment is a highlight segment, add the respective additional audio segment and a corresponding additional image segment to a highlight video.

15. The non-transitory, computer-readable storage medium of claim 1, wherein the executable instructions further cause the one or more processors to:
in accordance with a determination, based on the respective transcription, that another respective audio segment of the plurality of audio segments is not a highlight segment, discard the other respective audio segment and another corresponding image segment of the plurality of image segments.

16. The non-transitory, computer-readable storage medium of claim 1, wherein the respective transcription of each audio segment of the plurality of audio segments is generated by an automatic speech recognition machine-learning model, based on the respective audio segment of the plurality of audio segments.

17. The non-transitory, computer-readable storage medium of claim 1, wherein the head-wearable device is a pair of smart glasses.

18. The non-transitory, computer-readable storage medium of claim 1, wherein:
generating the respective transcription of each audio segment of the plurality of audio segments is performed by a multi-modal artificial intelligence (AI); and
the determination, based on the respective transcription, that the respective audio segment is a highlight segment is made by the multi-modal AI.

19. A method comprising:
in response to a capture command:
capturing image data at one or more cameras of a head-wearable device; and
capturing audio data at one or more microphones of the head-wearable device;
divide the image data into a plurality of image segments;
divide the audio data into a plurality of audio segments, each audio segment of the plurality of audio segments corresponding to a respective image segment of the plurality of image segments;
generate a respective transcription of each audio segment of the plurality of audio segments;
in accordance with a determination, based on the respective transcription, that a respective audio segment is a highlight segment, add the respective audio segment and a corresponding image segment to a highlight video.

20. A system comprising:
a head-wearable device including one or more cameras and one or more microphones;
one or more processors; and
a non-transitory, computer-readable storage medium including executable instructions that, when executed by the one or more processors, cause the one or more processors to:
in response to a capture command:
receive image data captured at the one or more cameras of the head-wearable device; and
receive audio data captured at the one or more microphones of the head-wearable device;
cause the image data to be divided into a plurality of image segments;
cause the audio data to be divided into a plurality of audio segments, each audio segment of the plurality of audio segments corresponding to a respective image segment of the plurality of image segments;
generate a respective transcription of each audio segment of the plurality of audio segments;
in accordance with a determination, based on the respective transcription, that a respective audio segment is a highlight segment, add the respective audio segment and a corresponding image segment to a highlight video.
